(19) United States
(12) Patent Application Publication
(10) Pub. No.: US 2015/0342943 A1
(43) Pub. Date: Dec. 3, 2015

(54) METHODS OF TREATING LIVER DISEASE

(71) Applicant: Gilead Sciences, Inc., Foster City, CA (US)

(72) Inventors: Jeffrey D. Bornstein, Cambridge, MA (US); David Breckenridge, San Mateo, CA (US); Satyajit Karnik, Fremont, CA (US); Victoria Smith, Burlingame, CA (US); Daniel B. Tumas, San Carlos, CA (US)

(21) Appl. No.: 14/725,730

(22) Filed: May 29, 2015

Related U.S. Application Data
(60) Provisional application No. 62/007,361, filed on Jun. 3, 2014.
(60) Provisional application No. 62/076,427, filed on Nov. 6, 2014.

(51) Int. Cl. A61K 31/4439; A61K 39/395

(52) U.S. Cl. CPC A61K 31/4439; A61K 39/3955

(57) ABSTRACT

The present disclosure relates to a method of preventing and/or treating liver disease comprising administering an ASK1 inhibitor, optionally in combination with a LOXL2 inhibitor, to a patient in need thereof.

This application claims the benefit and the priority of U.S. provisional patent application Ser. No. 62/007,361, filed Jun. 3, 2014, and U.S. provisional patent application Ser. No. 62/076,427, filed Nov. 6, 2014, both disclosures are incorporated herein by reference in their entireties.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 1051_P2C_SeqList. The text file is 14.1 KB, was created on Aug. 7, 2015, and is submitted electronically via EFS-Web.

### FIELD

The present disclosure relates to a method of preventing and/or treating liver disease comprising administering an ASK1 inhibitor, optionally in combination with a LOXL2 inhibitor, to a patient in need thereof.

### BACKGROUND

Liver disease is generally classified as acute or chronic based upon the duration of the disease. Liver disease may be caused by infection, injury, exposure to drugs or toxic compounds, alcohol, impurities in foods, and the abnormal build-up of normal substances in the blood, an autoimmune process, a genetic defect (such as haemochromatosis), or unknown cause(s). Common liver diseases include cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

Liver disease is a leading cause of death world wide. In particular, it has been seen that a diet high in fat damages the liver in ways that are surprisingly similar to hepatitis. The American Liver Foundation estimates that more than 20 percent of the population has non-alcoholic fatty liver disease (NAFLD). It is suggested that obesity, unhealthy diets, and sedentary lifestyles may contribute to the high prevalence of NAFLD. When left untreated, NAFLD can progess to non-alcoholic steatohepatitis (NASH), causing serious adverse effects. Once NASH is developed, it would cause the liver to swell and scar (i.e. cirrhosis) over time.

Although preliminary reports suggest positive lifestyle changes could prevent or reverse liver damage, there are no effective medical treatments for NAFLD. Accordingly, there remains a need to provide new effective pharmaceutical agents to treat liver diseases.

### SUMMARY

Disclosed herein is a method of treating and/or preventing liver disease in a patient in need thereof, comprising administering to the patient a therapeutically effective amount of an ASK1 inhibitor, optionally in combination with a therapeutically effective amount of a LOXL2 inhibitor. The liver disease can be any liver disease, including, but not limited to, chronic and/or metabolic liver diseases. In one embodiment, the liver disease is nonalcoholic fatty liver disease (NAFLD). In some embodiments, the liver disease is steatosis. In additional embodiments, the liver disease is liver fibrosis. In certain embodiment, the liver disease is nonalcoholic steatohepatitis (NASH). In other embodiment, the liver disease is primary sclerosing cholangitis (PSC).

In certain embodiments, provided herein is a method of treating and/or preventing nonalcoholic steatohepatitis (NASH) in a patient in need thereof, comprising administering to the patient a therapeutically effective amount of an ASK1 inhibitor, optionally in combination with a therapeutically effective amount of a LOXL2 inhibitor.

In other embodiments, provided herein is a method of treating and/or preventing liver fibrosis in a patient in need thereof, comprising administering to the patient a therapeutically effective amount of an ASK1 inhibitor, optionally in combination with a therapeutically effective amount of a LOXL2 inhibitor.

In still other embodiments, provided herein is a method of treating and/or preventing primary sclerosing cholangitis (PSC) in a patient in need thereof, comprising administering to the patient a therapeutically effective amount of an ASK1 inhibitor, optionally in combination with a therapeutically effective amount of a LOXL2 inhibitor.

In further embodiments, provided herein is a method of treating and/or preventing steatosis in a patient in need thereof, comprising administering to the patient a therapeutically effective amount of an ASK1 inhibitor, optionally in combination with a therapeutically effective amount of a LOXL2 inhibitor.

In the methods provided herein, the ASK1 inhibitor and the LOXL2 inhibitor can be coadministered. In such embodiments, the ASK1 inhibitor and the LOXL2 inhibitor can be administered together as a single pharmaceutical composition, or separately in more than one pharmaceutical composition. Accordingly, also provided herein is a pharmaceutical composition comprising a therapeutically effective amount of an ASK1 inhibitor and a therapeutically effective amount of a LOXL2 inhibitor.

Moreover, the application provides uses of the compounds in the manufacture of a medicament for the treatment of a liver disease. Also provided is a kit that includes an ASK1 inhibitor and optionally a LOXL2 inhibitor. The kit may further comprise a label and/or instructions for use of the ASK1 inhibitor, and optionally the LOXL2 inhibitor, in treating a liver disease in a human in need thereof. Further provided are articles of manufacture that include an ASK1 inhibitor, optionally a LOXL2 inhibitor, and a container. In one embodiment, the container may be a vial, jar, ampoule, preloaded syringe, or an intravenous bag.

### BRIEF DESCRIPTION OF THE DRAWINGS

FIG. 1 shows a comparison of the body weight of ASK1 inhibitor-treated subjects (Compound 1) on a fast-food diet (center trace) compared to controls (subjects on a normal diet (bottom trace) and a fast-food diet (top trace)).

FIG. 2 shows the daily food consumption (in grams) of ASK1 inhibitor-treated subjects (Compound 1) on a fast-food diet (bottom trace) compared to controls (subjects on a normal diet (top trace) and a fast-food diet (center trace)).

FIG. 3 shows the blood glucose levels (in mg/dL) of ASK1 inhibitor-treated subjects (Compound 1) on a fast-food diet at day 90, 180 and 270 compared to control subjects on a normal diet and subjects on a fast-food diet in the absence of an ASK1 inhibitor.

FIG. 4 shows the insulin levels (AUC via oral glucose tolerance test) of ASK1 inhibitor-treated subjects (Compound 1) a fast-food diet at day 90, 180 and 270 compared to control subjects on a normal diet and subjects on a fast-food diet in the absence of an ASK1 inhibitor.

FIG. 5 shows the insulin levels (in pg/dL) of ASK1 inhibitor-treated subjects (Compound 1) on a fast-food diet at day 90, 180 and 270 compared to control subjects on a normal diet and subjects on a fast-food diet in the absence of an ASK1 inhibitor.

FIGS. 6 and 7 show the liver function enzyme levels of ASK1 inhibitor-treated subjects (Compound 1) on a fast-food diet at day 90, 180 and 270 compared to control subjects on a normal diet and subjects on a fast-food diet in the absence of an ASK1 inhibitor. FIG. 6 shows the alanine aminotransferase (ALT) levels (in IU/L) and FIG. 7 shows the aspartate aminotransferase (AST) levels (in mg/dL).

FIG. 8 shows the serum cholesterol levels (in mg/dL) of ASK1 inhibitor-treated subjects (Compound 1) on a fast-food diet at day 90, 180 and 270 compared to control subjects on a normal diet and subjects on a fast-food diet in the absence of an ASK1 inhibitor.

FIG. 9 shows the percent collagen area (PCA) by quantitative morphometry at 180 days of ASK1 inhibitor-treated subjects (Compound 1) on a fast-food diet compared to control subjects on a normal diet and subjects on a fast-food diet in the absence of an ASK1 inhibitor.

FIG. 10 shows the liver collagen content at 180 days of ASK1 inhibitor-treated subjects (Compound 1) on a fast-food diet compared to control subjects on a normal diet and subjects on a fast-food diet in the absence of an ASK1 inhibitor. The liver collagen content is shown by relative hydroproline levels.

FIG. 11 shows the hepatic steatosis grade (x-axis) and fibrosis severity (F-score, y-axis) as assessed at 90 and 180 days ASK1 inhibitor-treated subjects (Compound 1) on a fast-food diet compared to subjects on a fast-food diet in the absence of an ASK1 inhibitor

FIG. 12 shows the synergistic effect of an ASK1 inhibitor and a LOXL2 inhibitor on hydroxy proline levels (as a predictor of liver collagen levels) in a fast food diet model at day 315 (i.e., end of treatment period) versus day 240 (i.e., start of treatment period).

FIGS. 13A and 13B show that the levels of steatosis and fibrosis in the control group on a normal diet,on the group on a fast-food diet without any treatment, and the group on a fast-food diet with Compound 1 treatment (P<0.001 in A and P<0.01 in B). (A) shows the levels of steatosis, and (B) shows the levels of fibrosis. The levels of fibrosis is shown by PSR area which is the area showing PSR (picrosirius red) staining. *** represents P value.

FIG. 14 show the blood glucose levels (in mg/dL) at day 90, 180, 270 and 360 in the control group on a normal diet,on the group on a fast-food diet without any treatment, and the group on a fast-food diet with Compound 1 treatment (P<0.01). ** represents P value.

FIG. 15 shows the insulin levels (AUC via oral glucose tolerance test) at day 90, 180, 270 and 360 in the control group on a normal diet,on the group on a fast-food diet without any treatment, and the group on a fast-food diet with Compound 1 treatment (P<0.001, P<0.0001). P<0.001 for day 90, P<0.0001 for day 180, 270, and 360.

FIG. 16 shows the insulin levels (in IU/L) at day 90, 180, 270 and 360 in the control group on a normal diet,on the group on a fast-food diet without any treatment, and the group on a fast-food diet with Compound 1 treatment (P<0.01). ** represents P value.

FIG. 17 shows the alanine aminotransferase (ALT) levels (in IU/L) at day 90, 180, 270 and 360 in the control group on a normal diet,on the group on a fast-food diet without any treatment, and the group on a fast-food diet with Compound 1 treatment (P<0.001). ** represents P value.

FIG. 18 shows the aspartate aminotransferase (AST) levels (in mg/dL) at day 90, 180, 270 and 360 in the control group on a normal diet,on the group on a fast-food diet without any treatment, and the group on a fast-food diet with Compound 1 treatment (P<0.001). ** represents P value.

FIG. 19 shows that the hypercholesterolemia at day 90, 180, 270 and 360 in the control group on a normal diet,on the group on a fast-food diet without any treatment, and the group on a fast-food diet with Compound 1 treatment (P<0.01, P<0.001). Hypercholesterolemia is shown by the cholesterol levels (in mg/dL).

FIG. 20 shows the glucose levels (AUC via oral glucose tolerance test) at day 90, 180, 270 and 360 in the control group on a normal diet, the group on a fast-food diet without any treatment, and the group on a fast-food diet with Compound 1 treatment (P<0.01). ** represents P value.

FIG. 21 shows that the fibrosis progression at day 90, 180, 270 and 360 in the control group on a normal diet,on the group on a fast-food diet without any treatment, and the group on a fast-food diet with Compound 1 treatment (P<0.0001). Fibrosis progression is shown by relative hydroxyproline. ** represents P value.

FIG. 22 shows that an ASK1 inhibitor (Compound 1) is efficacious in murine model of primary sclerosing cholangitis (P<0.0001). Compound 1 reduced liver fibrosis (i.e. efficacious) as determined by the levels of liver hydroxyproline and the area of tissue collagen in liver sections in a PSC murine model. ASK1i is ASK1 inhibitor (Compound 1); PCA is percentage of collagen area.

FIGS. 23A and 23B shows the (A) hydroxyproline synthesis (as a predictor of liver collagen levels) and (B) hepatic steatosis in a fast food diet model at day 315 (i.e., end of Compound 1 treatment period) versus day 240 (i.e., start of Compound 1 treatment period) (P<0.05). HYP is hydroxyproline.

FIGS. 24A and 24B show the rate of fibrillar collagen synthesis in a fast food diet model at day 315 (i.e., end of Compound 1 treatment period) versus day 240 (i.e., start of Compound 1 treatment period) (*P<0.05; **P<0.005). (A) shows the levels of soluable collagen synthesis, and (B) shows the levels of insoluable collagen synthesis.

FIGS. 25A and 25B show (A) the aspartate aminotransferase (AST) levels (in IU/L) and (B) the alanine aminotransferase (ALT) levels (in IU/L) at day 315 (i.e., end of treatment period) of the treatment starts at day 240 (i.e., start of treatment period). (P<0.05).

FIGS. 26A and 26B shows (A) fasting blood glucose and (B) insulin levels in at day 315 (i.e., end of Compound 1 treatment period) of the treatment starts at day 240 (i.e., start of Compound 1 treatment period) (P<0.001 in A and P<0.01 in B). The insulin levels in (B) refer to the fasting blood insulin levels.

FIGS. 27A and 27B shows (A) glucose metabolism and (B) insulin resistance at day 315 (i.e., end of Compound 1 treatment period) of the treatment starts at day 240 (i.e., start of Compound 1 treatment period) (P<0.001 in A and P<0.01 in B). The glucose metabolism in (A) is shown by AUC glucose, and the insulin resistance in (B) is shown by AUC insulin.

FIG. 28 shows the serum cholesterol levels (in mg/dL) at day 315 (i.e., end of Compound 1 treatment period) of the treatment starts at day 240 (i.e., start of Compound 1 treatment period) (P<0.05).

FIGS. 29A-29F shows (A) relative hepatic hydroxyproline, (B) total hepatic hydroxyproline, (C) alanine aminotransferase (ALT) levels (in U/l) in serum, (D) ALP (in U/l) in serum (E) portal venous pressure (PVP), and (F) relative liver weight in Mdr2^{-/-} BALB/c, treated with vehicle, iso (i.e. isotype antibody, an unrelated murine IgG1), AB0023 (anti-LOXL2 antibody), ASK1i (Compound 1), or combo (AB0023 and Compound 1) at 12 weeks (i.e. end of 6-week treatment period), end illustrates before 6-week treatment, and start illustrates after 6-week treatment (* P<0.05, ** P<0.01, *** P<0.001, ANOVA with Dunnett's post-test).

FIG. 30 shows the relative hydroxyproline levels in the groups, in normal diet or fast food diet, treated with vehicle (Ctrl), an unrelated control isotype antibody (IgG), Compound 1 (ASK1i), AB0023 (Anti-LOXL2), the combination of Compound 1 and AB0023 (ASK1i+anti-LOXL2), or the combination of Compound 1 and control antibody (ASK1i+IgG).

### DETAILED DESCRIPTION

### Definitions and General Parameters

As used in the present specification, the following terms and phrases are generally intended to have the meanings as set forth below, except to the extent that the context in which they are used indicates otherwise.

As used herein, the term “about” used in the context of quantitative measurements means the indicated amount ±10%, or alternatively the indicated amount ±5% or ±1%.

As referred to herein, an “ASK1 inhibitor” may be any agent that is capable of inactivating an apoptosis signal regulating kinase 1 (ASK1) protein. The agent may be a chemical compound or biological molecule (e.g., a protein or antibody). The ASK1 protein activity may be measured by several different methods. For example, the activity of an ASK1 protein may be determined based on the ability of the ASK1 protein to phosphorylate a substrate protein. Methods for identifying an ASK1 inhibitor are known (see, e.g., U.S. 2007/0276050 and U.S. 2011/0009410, both of which are incorporated herein by reference in their entirety). Exemplary ASK1 substrate proteins include MAPKK3, MAPKK4, MAPKK6, MAPKK7, or fragments thereof. The ASK1 protein activity may also be measured by the phosphorylation level of the ASK1 protein, for example, the phosphorylation level of a threonine residue in the ASK1 protein corresponding to threonine 838 (T838) of a human full-length ASK1 protein or threonine 845 (T845) of a mouse full-length ASK1 protein. For example, where the ASK1 protein comprises a full-length human ASK1 protein sequence, an ASK1 inhibitor may attenuate phosphorylation of T838 in the full-length human ASK1 protein sequence. A site specific antibody against human ASK1 T838 or mouse ASK1 T845 may be used to detect the phosphohorylation level.

As used herein, a “LOXL2 inhibitor” any agent that is capable of inactivating lysyl oxidase-like 2 (LOXL2) protein. The agent may be a chemical compound or biological molecule (e.g., a protein or antibody). The LOXL2 protein activity may be measured by several different methods (see, e.g., U.S. 2009/0053224 and U.S. 2011/0044907, both of which are incorporated herein by reference in their entirety). In certain embodiments, the LOXL2 inhibitor is an anti-LOXL2 antibody and antigen binding fragments thereof that bind to and/or inhibit LOXL2. In other embodiments, the LOXL2 inhibitor is the anti-LOXL2 antibody described in U.S. Pat. No. 8,461,303, U.S. 2012/0309020, U.S. 2013/0324705, and U.S. 2014/0079707, each of which are incorporated herein by reference in their entirety. The term “antibody” is used herein refers to a population of immunoglobulin molecules and/or immunologically active portions of immunoglobulin molecules, i.e., molecules that contain an antibody combining site or paratope. Thus, reference to an “antibody” also includes reference to any of the antigen binding fragments of antibodies. The term “antibody” also includes molecules which have been engineered through the use of molecular biological technique to include only portions of the native molecule as long as those molecules have the ability to bind a particular antigen or sequence of amino acids with the required specificity. Such alternative antibody molecules include classically known portions of the antibody molecules, single chain antibodies, and single chain binding molecules. Thus, it is used in the broadest sense and specifically covers monoclonal antibodies (including full length monoclonal antibodies), polyclonal antibodies, human antibodies, humanized antibodies, chimeric antibodies, diabodies, multispecific antibodies (e.g., bispecific antibodies), and antibody fragments including but not limited to single chain binding polypeptides, so long as they exhibit the desired biological activity. An antibody can be a humanized antibody. Humanized forms of non-human (e.g., murine) antibodies include, for example, chimeric immunoglobulins, immunoglobulin chains or fragments thereof (such as Fv, scFv, Fab, Fab′, F(ab′)2, single chain binding polypeptide, VH, VL, or other antigen-binding subsequences of antibodies) which contain minimal sequence derived from non-human immunoglobulin. Chimeric antibodies include those in which the heavy and light chain variable regions are combined with human constant regions (Fc). Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody can also contain at least a portion of an immunoglobulin constant region (Fc), typically that of a human immunoglobulin (Jones et al., Nature, 321:522-525 (1986); Riechmann et al., Nature, 332:323-329(1988); and Presta, Curr. Op. Struct. Biol. 2:593-596 (1992)). Methods for humanizing non-human antibodies are well known in the art. Generally, a humanized antibody has one or more amino acid residues introduced into it from a source that is non-human. These non-human amino acid residues are often referred to as “import” or “donor” residues, which are typically taken from an “import” or “donor” variable domain. Humanization can be essentially performed following the method of Winter and co-workers (Jones et al., Nature, 321:522 525 (1986); Riechmann et al., Nature, 332:323 327 (1988)); Verhoeyen et al. Science, 239:1534 1536 (1988)), by substituting rodent CDRs or CDR sequences for the corresponding sequences of a human antibody. Accordingly, such “humanized” antibodies include chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

“Antigen binding fragments” comprise a portion of an intact antibody, and can include the antigen binding or variable region of an intact antibody. Examples of antibody fragments include Fab, Fab′, F(ab′)2, Fv fragments, scFv fragments, diabodies, linear antibodies (Zapata et al., Protein Eng. 8(10): 1057-1062 (1995)), single-chain antibody molecules, single chain binding polypeptides, and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “binding” refers to a direct association between two molecules, due to, for example, covalent, electrostatic, hydrophobic, and ionic and/or hydrogen-bond interactions, including interactions such as salt bridges and water bridges. The term “specific binding” is applicable to a situation in which an antibody or antigen binding fragment thereof does not show any significant binding to molecules other than its epitope. In one embodiment, an antibody or antigen binding fragment thereof specifically binds to a human LOX or to human LOXL2 with a dissociation constant Kd equal to or lower than about 100 nM, lower than about 10 nM, lower than about 1 nM, lower than about 0.5 nM, lower than about 0.1 nM, lower than about 0.01 nM, or lower than about 0.005 nM measured at a temperature of about 4° C., 25° C., 37° C. or 42° C.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

“Homology” describes a mathematically based comparison of sequence similarities which is used to identify genes or proteins with similar functions or motifs. The nucleic acid (nucleotide, oligonucleotide) and amino acid (protein) sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST amino acid searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (see, ncbi.nlm.nih.gov).

“Identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “fast-food diet” refers to a diet which is higher in one or more of fat, cholesterol, sugar, and total calories as compared to the recommended daily amount for a given subject as determined in the art (e.g., as determined by the U.S. Food and Drug Administration).

The term “normal diet” refers to a diet which does not exceed the recommended daily amount of fat, cholesterol, sugar, or total calories for a given subject as determined in the art (e.g., as determined by the U.S. Food and Drug Administration).

The terms “synergy” or “synergistic effect(s)” refer to the effect achieved when the active ingredients used together is greater than the sum of the effects that results from using the compounds separately or greater than the additive effects resulted from the compound alone. In certain embodiments, a synergistic effect may be attained when the compounds are administered or delivered separately, e.g., in separate tablets, pills or capsules, or by different injections in separate syringes. As shown in the Examples, the administration of LOXL2 inhibitor and ASK1 inhibitor provide unexpected synergy or synergistic effect(s).

The term “pharmaceutically acceptable salt” refers to salts of pharmaceutical compounds e.g. compound of formula (I) that retain the biological effectiveness and properties of the underlying compound, and which are not biologically or otherwise undesirable. There are acid addition salts and base addition salts. Pharmaceutically acceptable acid addition salts may be prepared from inorganic and organic acids.

Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangibly and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt of formula (I) for the purpose of:

(i) delaying the onset of a disease, that is, causing the clinical symptoms of the disease not to develop or delaying the development thereof;

(ii) inhibiting the disease, that is, arresting the development of clinical symptoms; and/or

(iii) relieving the disease, that is, causing the regression of clinical symptoms or the severity thereof. In some embodiments, the term “treatment” or “treating” also means promoting resolution of the disease or promoting the regression of clinical symptoms or the severity of the disease or the symptoms.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human. In certain embodiments, the subject is a patient having liver disease. In additional embodiments, the subject is a patient having or suspected to have non-alcoholic steatohepatitis (NASH). In one embodiment, the subject is a patient is having or suspected to have primary sclerosing cholangitis (PSC). In other embodiment, the subject is a patient having or suspected to have primary biliary cirrhosis (PBC). In some other embodiment, the subject is a patient having or suspected to have non-alcoholic fatty liver disease (NAFLD). In some other embodiment, the subject is a patient having or suspected to have steatosis or fatty liver. The terms “subject in need thereof” or “patient in need thereof” refer to a subject or a patient who may have, is diagnosized, or is suspected to have diseases, or disorders, or conditions that would benefit from the treatment described herein. In certain embodiments, the subject or patient who (i) has not received any treatment, (ii) has received prior treatment and is not responsive or did not exhibit improvement, or (iii) is relapse or resistance to prior treatment.

The term “alkyl” refers to a monoradical branched or unbranched saturated hydrocarbon chain having from 1 to 20 carbon atoms, or from 1 to 15 carbon atoms, or from 1 to 10 carbon atoms, or from 1 to 8 carbon atoms, or from 1 to 6 carbon atoms, or from 1 to 4 carbon atoms. This term is exemplified by groups such as methyl, ethyl, n-propyl, iso-propyl, n-butyl, iso-butyl, t-butyl, n-hexyl, n-decyl, tetradecyl, and the like.

The term “substituted alkyl” refers to:

1) an alkyl group as defined above, having 1, 2, 3, 4 or 5 substituents, (in some embodiments, 1, 2 or 3 substituents) selected from the group consisting of alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or

2) an alkyl group as defined above that is interrupted by 1-10 atoms (e.g. 1, 2, 3, 4 or 5 atoms) independently chosen from oxygen, sulfur and NR^{a}, where R^{a} is chosen from hydrogen, alkyl, cycloalkyl, alkenyl, cycloalkenyl, alkynyl, aryl, heteroaryl and heterocyclyl. All substituents may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2; or

3) an alkyl group as defined above that has both 1, 2, 3, 4 or 5 substituents as defined above and is also interrupted by 1-10 atoms (e.g. 1, 2, 3, 4 or 5 atoms) as defined above.

The term “lower alkyl” refers to a monoradical branched or unbranched saturated hydrocarbon chain having 1, 2, 3, 4, 5 or 6 carbon atoms. This term is exemplified by groups such as methyl, ethyl, n-propyl, iso-propyl, n-butyl, iso-butyl, t-butyl, n-hexyl, and the like.

The term “substituted lower alkyl” refers to lower alkyl as defined above having 1 to 5 substituents (in some embodiments, 1, 2 or 3 substituents), as defined for substituted alkyl or a lower alkyl group as defined above that is interrupted by 1, 2, 3, 4 or 5 atoms as defined for substituted alkyl or a lower alkyl group as defined above that has both 1, 2, 3, 4 or 5 substituents as defined above and is also interrupted by 1, 2, 3, 4 or 5 atoms as defined above.

The term “alkylene” refers to a diradical of a branched or unbranched saturated hydrocarbon chain, in some embodiments, having from 1 to 20 carbon atoms (e.g. 1-10 carbon atoms or 1, 2, 3, 4, 5 or 6 carbon atoms). This term is exemplified by groups such as methylene (—CH₂—), ethylene (—CH₂CH₂—), the propylene isomers (e.g., —CH₂CH₂CH₂— and —CH(CH₃)CH₂—), and the like.

The term “lower alkylene” refers to a diradical of a branched or unbranched saturated hydrocarbon chain, in some embodiments, having 1, 2, 3, 4, 5 or 6 carbon atoms.

The term “substituted alkylene” refers to an alkylene group as defined above having 1 to 5 substituents (in some embodiments, 1, 2 or 3 substituents) as defined for substituted alkyl.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. Such aralkyl groups are exemplified by benzyl, phenylethyl, 3-(4-methoxyphenyl)propyl, and the like.

The term “aralkyloxy” refers to the group —O-aralkyl. “Optionally substituted aralkyloxy” refers to an optionally substituted aralkyl group covalently linked to an optionally substituted alkylene group. Such aralkyl groups are exemplified by benzyloxy, phenylethyloxy, and the like.

The term “alkenyl” refers to a monoradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds. In some embodiments, alkenyl groups include ethenyl (or vinyl, i.e. —CH═CH₂), 1-propylene (or allyl, i.e. —CH₂CH═CH₂), isopropylene (—C(CH₃)═CH₂), and the like.

The term “lower alkenyl” refers to alkenyl as defined above having from 2 to 6 carbon atoms.

The term “substituted alkenyl” refers to an alkenyl group as defined above having 1 to 5 substituents (in some embodiments, 1, 2 or 3 substituents) as defined for substituted alkyl.

The term “alkenylene” refers to a diradical of a branched or unbranched unsaturated hydrocarbon group having from 2 to 20 carbon atoms (in some embodiments, from 2 to 10 carbon atoms, e.g. 2 to 6 carbon atoms) and having from 1 to 6 carbon-carbon double bonds, e.g. 1, 2 or 3 carbon-carbon double bonds.

The term “alkynyl” refers to a monoradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds. In some embodiments, alkynyl groups include ethynyl (—C≡CH), propargyl (or propynyl, i.e. —C≡CCH₃), and the like.

The term “substituted alkynyl” refers to an alkynyl group as defined above having 1 to 5 substituents (in some embodiments, 1, 2 or 3 substituents) as defined for substituted alkyl.

The term “alkynylene” refers to a diradical of an unsaturated hydrocarbon, in some embodiments, having from 2 to 20 carbon atoms (in some embodiments, from 2 to 10 carbon atoms, e.g. 2 to 6 carbon atoms) and having from 1 to 6 carbon-carbon triple bonds e.g. 1, 2 or 3 carbon-carbon triple bonds.

The term “hydroxy” or “hydroxyl” refers to a group —OH.

The term “alkoxy” refers to the group R—O—, where R is alkyl or —Y—Z, in which Y is alkylene and Z is alkenyl or alkynyl, where alkyl, alkenyl and alkynyl are as defined herein. In some embodiments, alkoxy groups are alkyl-O— and includes, by way of example, methoxy, ethoxy, n-propoxy, iso-propoxy, n-butoxy, tert-butoxy, sec-butoxy, n-pentoxy, n-hexyloxy, 1,2-dimethylbutoxy, and the like.

The term “lower alkoxy” refers to the group R—O— in which R is optionally substituted lower alkyl. This term is exemplified by groups such as methoxy, ethoxy, n-propoxy, iso-propoxy, n-butoxy, iso-butoxy, t-butoxy, n-hexyloxy, and the like.

The term “substituted alkoxy” refers to the group R—O—, where R is substituted alkyl or —Y—Z, in which Y is substituted alkylene and Z is substituted alkenyl or substituted alkynyl, where substituted alkyl, substituted alkenyl and substituted alkynyl are as defined herein.

The term “C₁₋₃ haloalkyl” refers to an alkyl group having from 1 to 3 carbon atoms covalently bonded to from 1 to 7, or from 1 to 6, or from 1 to 3, halogen(s), where alkyl and halogen are defined herein. In some embodiments, C₁₋₃ haloalkyl includes, by way of example, trifluoromethyl, difluoromethyl, fluoromethyl, 2,2,2-trifluoroethyl, 2,2-difluoroethyl, 2-fluoroethyl, 3,3,3-trifluoropropyl, 3,3-difluoropropyl, 3-fluoropropyl.

The term “C₁₋₃ hydroxyalkyl” refers to an alkyl group having a carbon atom covalently bonded to a hydroxy, where alkyl and hydroxy are defined herein. In some embodiments, C₁₋₃ hydroxyalkyl includes, by way of example, 2-hydroxyethyl.

The term “C₁₋₃ cyanoalkyl” refers to an alkyl group having a carbon atom covalently bonded to a cyano, where alkyl and cyano are defined herein. In some embodiments, C₁₋₃ cyanoalkyl includes, by way of example, 2-cyanoethyl.

The term “cycloalkyl” refers to cyclic alkyl groups of from 3 to 20 carbon atoms, or from 3 to 10 carbon atoms, having a single cyclic ring or multiple condensed rings. Such cycloalkyl groups include, by way of example, single ring structures such as cyclopropyl, cyclobutyl, cyclopentyl, cyclooctyl and the like or multiple ring structures such as adamantanyl and bicyclo[2.2.1]heptanyl or cyclic alkyl groups to which is fused an aryl group, for example indanyl, and the like, provided that the point of attachment is through the cyclic alkyl group.

The term “cycloalkenyl” refers to cyclic alkyl groups of from 3 to 20 carbon atoms having a single cyclic ring or multiple condensed rings and having at least one double bond and in some embodiments, from 1 to 2 double bonds.

The terms “substituted cycloalkyl” and “substituted cycloalkenyl” refer to cycloalkyl or cycloalkenyl groups having 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. The term “substituted cycloalkyl” also includes cycloalkyl groups wherein one or more of the annular carbon atoms of the cycloalkyl group has an oxo group bonded thereto. In addition, a substituent on the cycloalkyl or cycloalkenyl may be attached to the same carbon atom as, or is geminal to, the attachment of the substituted cycloalkyl or cycloalkenyl to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “cycloalkoxy” refers to the group cycloalkyl-O—.

The term “substituted cycloalkoxy” refers to the group substituted cycloalkyl-O—.

The term “cycloalkenyloxy” refers to the group cycloalkenyl-O—.

The term “substituted cycloalkenyloxy” refers to the group substituted cycloalkenyl-O—.

The term “aryl” refers to an aromatic carbocyclic group of 6 to 20 carbon atoms having a single ring (e.g., phenyl) or multiple rings (e.g., biphenyl) or multiple condensed (fused) rings (e.g., naphthyl, fluorenyl and anthryl). In some embodiments, aryls include phenyl, fluorenyl, naphthyl, anthryl, and the like.

Unless otherwise constrained by the definition for the aryl substituent, such aryl groups can optionally be substituted with 1, 2, 3, 4 or 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aryloxy” refers to the group aryl-O— wherein the aryl group is as defined above, and includes optionally substituted aryl groups as also defined above. The term “arylthio” refers to the group R—S—, where R is as defined for aryl.

The term “heterocyclyl,” “heterocycle,” or “heterocyclic” refers to a monoradical saturated group having a single ring or multiple condensed rings, having from 1 to 40 carbon atoms and from 1 to 10 hetero atoms, and from 1 to 4 heteroatoms, selected from nitrogen, sulfur, phosphorus, and/or oxygen within the ring. In some embodiments, the heterocyclyl,” “heterocycle,” or “heterocyclic” group is linked to the remainder of the molecule through one of the heteroatoms within the ring.

Unless otherwise constrained by the definition for the heterocyclic substituent, such heterocyclic groups can be optionally substituted with 1 to 5 substituents (in some embodiments, 1, 2 or 3 substituents), selected from the group consisting of alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)— heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. In addition, a substituent on the heterocyclic group may be attached to the same carbon atom as, or is geminal to, the attachment of the substituted heterocyclic group to the 6,7-ring system. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Examples of heterocyclics include tetrahydrofuranyl, morpholino, piperidinyl, and the like.

The term “heterocycloxy” refers to the group —O-heterocyclyl.

The term “heteroaryl” refers to a group comprising single or multiple rings comprising 1 to 15 carbon atoms and 1 to 4 heteroatoms selected from oxygen, nitrogen and sulfur within at least one ring. The term “heteroaryl” is generic to the terms “aromatic heteroaryl” and “partially saturated heteroaryl”. The term “aromatic heteroaryl” refers to a heteroaryl in which at least one ring is aromatic, regardless of the point of attachment. Examples of aromatic heteroaryls include pyrrole, thiophene, pyridine, quinoline, pteridine.

The term “partially saturated heteroaryl” refers to a heteroaryl having a structure equivalent to an underlying aromatic heteroaryl which has had one or more double bonds in an aromatic ring of the underlying aromatic heteroaryl saturated. Examples of partially saturated heteroaryls include dihydropyrrole, dihydropyridine, chroman, 2-oxo-1,2-dihydropyridin-4-yl, and the like.

Unless otherwise constrained by the definition for the heteroaryl substituent, such heteroaryl groups can be optionally substituted with 1 to 5 substituents (in some embodiments, 1, 2 or 3 substituents) selected from the group consisting alkyl, alkenyl, alkynyl, alkoxy, cycloalkyl, cycloalkenyl, cycloalkoxy, cycloalkenyloxy, acyl, acylamino, acyloxy, amino, substituted amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —S(O)-alkyl, —S(O)-cycloalkyl, —S(O)-heterocyclyl, —S(O)-aryl, —S(O)-heteroaryl, —S(O)₂-alkyl, —S(O)₂-cycloalkyl, —S(O)₂-heterocyclyl, —S(O)₂-aryl and —S(O)₂-heteroaryl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2. Such heteroaryl groups can have a single ring (e.g., pyridyl or furyl) or multiple condensed rings (e.g., indolizinyl, benzothiazole or benzothienyl). Examples of nitrogen heterocyclyls and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, and the like as well as N-alkoxy-nitrogen containing heteroaryl compounds.

The term “heteroaryloxy” refers to the group heteroaryl-O—.

The term “amino” refers to the group —NH₂.

The term “substituted amino” refers to the group —NRR where each R is independently selected from the group consisting of hydrogen, alkyl, cycloalkyl, aryl, heteroaryl and heterocyclyl provided that both R groups are not hydrogen or a group —Y—Z, in which Y is optionally substituted alkylene and Z is alkenyl, cycloalkenyl or alkynyl. Unless otherwise constrained by the definition, all substituents may optionally be further substituted by 1, 2 or 3 substituents chosen from alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkyl amine” refers to R—NH₂ in which R is optionally substituted alkyl.

The term “dialkyl amine” refers to R—NHR in which each R is independently an optionally substituted alkyl.

The term “trialkyl amine” refers to NR₃ in which each R is independently an optionally substituted alkyl.

The term “cyano” refers to the group —CN.

The term “azido” refers to a group —N

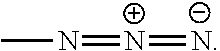

The term “keto” or “oxo” refers to a group ═O.

The term “carboxy” refers to a group —C(O)—OH.

The term “ester” or “carboxyester” refers to the group —C(O)OR, where R is alkyl, cycloalkyl, aryl, heteroaryl or heterocyclyl, which may be optionally further substituted by alkyl, alkoxy, halogen, CF₃, amino, substituted amino, cyano or —S(O)ₙR^{a}, in which R^{a} is alkyl, aryl or heteroaryl and n is 0, 1 or 2.

The term “acyl” denotes the group —C(O)R, in which R is hydrogen, alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “carboxyalkyl” refers to the groups —C(O)O-alkyl or —C(O)O-cycloalkyl, where alkyl and cycloalkyl are as defined herein, and may be optionally further substituted by alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonyl” refers to the group —C(O)NRR where each R is independently hydrogen, alkyl, cycloalkyl, aryl, heteroaryl, or heterocyclyl, or where both R groups are joined to form a heterocyclic group (e.g., morpholino). Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acyloxy” refers to the group —OC(O)—R, in which R is alkyl, cycloalkyl, heterocyclyl, aryl or heteroaryl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “acylamino” refers to the group —NRC(O)R where each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “alkoxycarbonylamino” refers to the group —N(R^{d})C(O)OR in which R is alkyl and R^{d} is hydrogen or alkyl. Unless otherwise constrained by the definition, each alkyl may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “aminocarbonylamino” refers to the group —NR^{c}C(O)NRR, wherein R^{c} is hydrogen or alkyl and each R is hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “thiol” refers to the group —SH.

The term “thiocarbonyl” refers to a group ═S.

The term “alkylthio” refers to the group —S-alkyl.

The term “substituted alkylthio” refers to the group —S-substituted alkyl.

The term “heterocyclylthio” refers to the group —S-heterocyclyl.

The term “arylthio” refers to the group —S-aryl.

The term “heteroarylthiol” refers to the group —S-heteroaryl wherein the heteroaryl group is as defined above including optionally substituted heteroaryl groups as also defined above.

The term “sulfoxide” refers to a group —S(O)R, in which R is alkyl, cycloalkyl, heterocyclyl, aryl or heteroaryl. “Substituted sulfoxide” refers to a group —S(O)R, in which R is substituted alkyl, substituted cycloalkyl, substituted heterocyclyl, substituted aryl or substituted heteroaryl, as defined herein.

The term “sulfone” refers to a group —S(O)₂R, in which R is alkyl, cycloalkyl, heterocyclyl, aryl or heteroaryl. “Substituted sulfone” refers to a group —S(O)₂R, in which R is substituted alkyl, substituted cycloalkyl, substituted heterocyclyl, substituted aryl or substituted heteroaryl, as defined herein.

The term “aminosulfonyl” refers to the group —S(O)₂NRR, wherein each R is independently hydrogen, alkyl, cycloalkyl, aryl, heteroaryl or heterocyclyl. Unless otherwise constrained by the definition, all substituents may optionally be further substituted by 1, 2 or 3 substituents selected from the group consisting of alkyl, alkenyl, alkynyl, carboxy, carboxyalkyl, aminocarbonyl, hydroxy, alkoxy, halogen, CF₃, amino, substituted amino, cyano, cycloalkyl, heterocyclyl, aryl, heteroaryl, and —S(O)ₙR^{a}, in which R^{a} is alkyl, aryl or heteroaryl and n is 0, 1 or 2.

The term “hydroxyamino” refers to the group —NHOH.

The term “alkoxyamino” refers to the group —NHOR in which R is optionally substituted alkyl.

The term “halogen” or “halo” refers to fluoro, bromo, chloro and iodo.

“Optional” or “optionally” means that the subsequently described event or circumstance may or may not occur, and that the description includes instances where said event or circumstance occurs and instances in which it does not.

A “substituted” group includes embodiments in which a monoradical substituent is bound to a single atom of the substituted group (e.g. forming a branch), and also includes embodiments in which the substituent may be a diradical bridging group bound to two adjacent atoms of the substituted group, thereby forming a fused ring on the substituted group.

Where a given group (moiety) is described herein as being attached to a second group and the site of attachment is not explicit, the given group may be attached at any available site of the given group to any available site of the second group. For example, a “lower alkyl-substituted phenyl”, where the attachment sites are not explicit, may have any available site of the lower alkyl group attached to any available site of the phenyl group. In this regard, an “available site” is a site of the group at which a hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to -substituted aryl-(substituted aryl)-substituted aryl.

### Liver Diseases

Liver diseases are acute or chronic damages to the liver based in the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis, including both viral and alcoholic hepatitis. Additional example of liver diseases includes, but are not limited to, primary sclerosing cholangitis (PSC).

Non-alcoholic fatty liver disease (NAFLD) is the build up of extra fat in liver cells that is not caused by alcohol. NAFLD may cause the liver to swell (i.e. steatohepatitis), which in turn may cause scarring (i.e. cirrhosis) over time and may lead to liver cancer or liver failure. NAFLD is characterized by the accumulation of fat in hepatocyes and is often associated with some aspects of metabolic syndrome (e.g. type 2 diabetes mellitus, insulin resistance, hyperlipidemia, hypertension). The frequency of this disease has become increasingly common due to consumption of carbohydrate-rich and high fat diets. A subset (˜20%) of NAFLD patients develop nonalcoholic steatohepatitis (NASH).

NASH, a subtype of fatty liver disease, is the more severe form of NAFLD. It is characterized by macrovesicular steatosis, balloon degeneration of hepatocytes, and/or inflammation ultimately leading to hepatic scarring (i.e. fibrosis). Patients diagnosed with NASH may progress to advanced stage liver fibrosis and eventually cirrhosis. Once NASH is developed, it could cause the liver to undergo destructive remodeling leading to scarring (i.e. cirrhosis) over time. The current treatment for cirrhotic NASH patients with end-stage disease is liver transplant.

A study has shown that a significant proportion of diagnosed NASH patients (39%) have not had a liver biopsy to confirm the diagnosis. A greater proportion of diagnosed NASH patients have metabolic syndrome parameters than what is reported in the literature (type-II diabetes mellitus 54%, Obesity 71%, metabolic syndrome 59%). 82% of physicians use a lower threshold value to define significant alcohol consumption compared with practice guideline recommendations. 88% of physicians prescribe some form of pharmacologic treatment for NASH (Vit E: prescribed to 53% of NASH patients, statins: 57%, metformin: 50%). Therefore, the vast majority of patients are prescribed medications despite a lack of a confirmed diagnosis or significant data to support the intervention and alcohol thresholds to exclude NASH are lower than expected.

While the mechanism or cause of NASH is unclear, the diagnosis criteria for NASH have been established. NASH may be the metabolic syndrome which may be characterized by the impact of obesity, insulin resistance, and/or hypercholesterolemia in the liver. Without being bound to any hypothesis, NASH may be resulted from the setting of steatosis and metabolic dysfunction, increased oxidative stress and the generation of reactive oxygen species (ROS), which may mediate the inflammatory changes in the liver (steatohepatitis) with progressive liver fibrosis (Koek et al., Clin. Chim. Acta, 412: 1297-1305 (2011); Sumida et al., Free Radical Research, 47 (11):869-880 (2013)).

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver fibrosis is the excessive accumulation of extracellular matrix proteins, including collagen, that occurs in most types of chronic liver diseases. Advanced liver fibrosis results in cirrhosis, liver failure, and portal hypertension and often requires liver transplantation. In some cases, advanced liver fibrosis may result in liver cancer.

### Methods

Disclosed herein is a method of treating and/or preventing liver disease in a patient in need thereof, comprising administering to the patient a therapeutically effective amount of an ASK1 inhibitor, optionally in combination with a therapeutically effective amount of a LOXL2 inhibitor. The presence of active liver disease can be detected by the existence of elevated enzyme levels in the blood. Specifically, blood levels of alanine aminotransferase (ALT) and aspartate aminotransferase (AST), above clinically accepted normal ranges, are known to be indicative of on-going liver damage. Additionally, blood bilirubin levels or other liver enzymes may be used as detection or diagnostic criteria. Routine monitoring of liver disease patients for blood levels of ALT and AST is used clinically to measure progress of the liver disease while on medical treatment. Reduction of elevated ALT and AST to within the accepted normal range is taken as clinical evidence reflecting a reduction in the severity of the patients on-going liver damage.

The results of the present application indicate that ASK1 may be involved in fibrogenesis and liver injury, and that ASK1 inhibitors may inhibit, prevent, reduce, or reverse liver fibrogenesis. This suggest that ASK1 inhibitors, such as Compounds 1-3, may be an anti-fibrotic agent that would have therapeutic or prophylactic effects for treating liver fibrosis such as NASH or PSC. Also, the results described herein suggest that treatment with ASK1 inhibitor alone would lead to improvements in metabolic parameters associated with NASH.

Moreover, the results of the present application indicate that LOXL2 inhibitors, such as anti-LOXL2 antibodies AB0023 and AB0024 described in U.S. Pat. No. 8,461,303, may inhibit, prevent, or reduce the cross-linking of hepatic collagen, liver fibrogenesis, and/or reversal of fibrosis. The present application suggests that, under certain conditions, the combination of ASK1 inhibitor and LOXL2 inhibitor would inhibit, reduce, prevent, or reverse biliary fibrosis and portal hypertension. Without being bound to any hypothesis, a combination therapy comprising an ASK1 inhibitor and a LOXL2 inhibitor would impact non-overlapping profibrogenic signal transduction pathways, where LOXL2 would be involved in crosslinking of fibrillar collagen and/or activation of pathologic fibroblasts (such as hepatic stellate cells). Accordingly, the combination therapy comprising an ASK1 inhibitor (such as Compound 1, Compound 2, and Compound 3) and a LOXL2 inhibor (such as AB0023 and AB0024) would provide potential therapeutic effects to liver disease.

In certain embodiments, the liver disease is a chronic liver disease. Chronic liver diseases involve the progressive destruction and regeneration of the liver parenchyma, leading to fibrosis and cirrhosis. In general, chronic liver diseases can be caused by viruses (such as hepatitis B, hepatitis C, cytomegalovirus (CMV), or Epstein Barr Virus (EBV)), toxic agents or drugs (such as alcohol, methotrexate, or nitrofurantoin), a metabolic disease (such as non-alcoholic fatty liver disease (NAFLD), non-alcoholic steatohepatitis (NASH), haemochromatosis, or Wilson's Disease), an autoimmune disease (such as Autoimmune Chronic Hepatitis, Primary Biliary Cirrhosis, or Primary Sclerosing Cholangitis), or other causes (such as right heart failure). In one embodiment, the present application provides a method of treating liver fibrosis. In some embodiment, the present application provides a method of treating non-alcoholic steatohepatitis (NASH). In certain embodiment, the present application provides a method of treating primary sclerosing cholangitis (PSC).

In one embodiment, provided herein is a method for reducing the level of cirrhosis. In one embodiment, cirrhosis is characterized pathologically by loss of the normal microscopic lobular architecture, with fibrosis and nodular regeneration. Methods for measuring the extent of cirrhosis are well known in the art. In one embodiment, the level of cirrhosis is reduced by about 5% to about 100%. In one embodiment, the level of cirrhosis is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

In certain embodiments, the liver disease is a metabolic liver disease. In one embodiment, the liver disease is non-alcoholic fatty liver disease (NAFLD). NAFLD is associated with insulin resistance and metabolic syndrome (obesity, combined hyperlipidemia, diabetes mellitus (type II) and high blood pressure). NAFLD is considered to cover a spectrum of disease activity, and begins as fatty accumulation in the liver (hepatic steatosis).

It has been shown that both obesity and insulin resistance probably play a strong role in the disease process of NAFLD. In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. NAFLD has also been linked to the consumption of soft drinks through the presence of high fructose corn syrup which may cause increased deposition of fat in the abdomen, although the consumption of sucrose shows a similar effect (likely due to its breakdown into fructose). Genetics has also been known to play a role, as two genetic mutations for this susceptibility have been identified.

If left untreated, NAFLD can develop into non-alcoholic steatohepatitis (NASH), which is the most extreme form of NAFLD, a state in which steatosis is combined with inflammation and fibrosis. NASH is regarded as a major cause of cirrhosis of the liver of unknown cause. Accordingly, provided herein is a method of treating and/or preventing nonalcoholic steatohepatitis (NASH) in a patient in need thereof, comprising administering to the patient a therapeutically effective amount of an ASK1 inhibitor, optionally in combination with a therapeutically effective amount of a LOXL2 inhibitor.

Also provided herein is a method of treating and/or preventing liver fibrosis in a patient in need thereof, comprising administering to the patient a therapeutically effective amount of an ASK1 inhibitor, optionally in combination with a therapeutically effective amount of a LOXL2 inhibitor. Liver fibrosis is the excessive accumulation of extracellular matrix proteins including collagen that occurs in most types of chronic liver diseases. In certain embodiments, advanced liver fibrosis results in cirrhosis and liver failure. Methods for measuring liver histologies, such as changes in the extent of fibrosis, lobular hepatitis, and periportal bridging necrosis, are well known in the art.

In one embodiment, the level of liver fibrosis, which is the formation of fibrous tissue, fibroid or fibrous degeneration, is reduced by more that about 90%. In one embodiment, the level of fibrosis, which is the formation of fibrous tissue, fibroid or fibrous degeneration, is reduced by at least about 90%, at least about 80%, at least about 70%, at least about 60%, at least about 50%, at least about 40%, at least about 30%, at least about 20%, at least about 10%, at least about 5% or at least about 2%.

In one embodiment, the compounds provided herein reduce the level of fibrogenesis in the liver. Liver fibrogenesis is the process leading to the deposition of an excess of extracellular matrix components in the liver known as fibrosis. It is observed in a number of conditions such as chronic viral hepatitis B and C, alcoholic liver disease, drug-induced liver disease, hemochromatosis, auto-immune hepatitis, Wilson disease, primary biliary cirrhosis, sclerosing cholangitis, liver schistosomiasis and others. In one embodiment, the level of fibrogenesis is reduced by more that about 90%. In one embodiment, the level of fibrogenesis is reduced by at least about 90%, at least about 80%, at least about 70%, at least about 60%, at least about 50%, at least 40%, at least about 30%, at least about 20%, at least about 10%, at least about 5% or at least 2%.

In still other embodiments, provided herein is a method of treating and/or preventing primary sclerosing cholangitis (PSC) in a patient in need thereof, comprising administering to the patient a therapeutically effective amount of an ASK1 inhibitor, optionally in combination with a therapeutically effective amount of a LOXL2 inhibitor.

In some other embodiments, a method is provided for providing a prophalatic treatment of liver disease (including chronic liver disease, a metabolic liver disease, nonalcoholic fatty liver disease), nonalcoholic steatohepatitis (NASH), or liver fibrosis primary sclerosing cholangitis (PSC) in a patient in need thereof, comprising administering to the patient a therapeutically effective amount of an ASK1 inhibitor, optionally in combination with a therapeutically effective amount of a LOXL2 inhibitor. In certain other embodiments, a method is provided for providing prophalatic treatment of liver disease (including chronic liver disease, a metabolic liver disease, nonalcoholic fatty liver disease), nonalcoholic steatohepatitis (NASH), or liver fibrosis primary sclerosing cholangitis (PSC) in a patient in need thereof, comprising administering to the patient a therapeutically effective amount of an ASK1 inhibitor. In some embodiment, the prophalatic treatment is provided to the patients having NASH or PSC. In some other embodiment, the effect of prophalatic treatment may be determined by steatosis, fibrosis progession, fasting blood glucose levels, AUC insulin levels, fasting insulin levels, ALT levels, AST levels, cholesterol levels, AUC glucose levels, relative hydroxyproline levels, fibrillar collagen synthesis, and/or body weight.

In certain embodiments, a method is provided for treating pre-existing abnormal levels of steatosis, fibrosis progession, fasting blood glucose, AUC insulin, fasting insulin, ALT, AST, cholesterol, AUC glucose, relative hydroxyproline, fibrillar collagen synthesis, and/or body weight in a patient in need thereof, comprising administering to the patient a therapeutically effective amount of an ASK1 inhibitor. The abnormal levels may be determined by the levels that are higher than those detected in healthy individuals. In certain other embodiment, the abnormal levels of steatosis, fibrosis progession, fasting blood glucose, AUC insulin, fasting insulin, ALT, AST, cholesterol, AUC glucose, relative hydroxyproline, fibrillar collagen synthesis, and/or body weight are associated with type 2 diabetes mellitus. Methods for measuring the levels or extent of steatosis, fibrosis progession, fasting blood glucose, AUC insulin, fasting insulin, ALT, AST, cholesterol, AUC glucose, relative hydroxyproline, fibrillar collagen synthesis, and/or body weight are well known in the art. In one embodiment, the level or extent of steatosis, fibrosis progession, fasting blood glucose, AUC insulin, fasting insulin, ALT, AST, cholesterol, AUC glucose, relative hydroxyproline, fibrillar collagen synthesis, and/or body weight would be reduced by about 5% to about 100%, at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100%. In other embodiment, the level or extent of steatosis or fatty liver would be reduced by about 5% to about 100%, at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100%.

In further embodiment, provided herein is a method of treating and/or preventing metabolic syndrome or type 2 diabetes mellitus in a patient in need thereof, comprising administering to the patient a therapeutically effective amount of an ASK1 inhibitor, optionally in combination with a therapeutically effective amount of a LOXL2 inhibitor. In further embodiment, provided herein is a method of treating and/or preventing insulin resistance in a patient in need thereof, comprising administering to the patient a therapeutically effective amount of an ASK1 inhibitor, optionally in combination with a therapeutically effective amount of a LOXL2 inhibitor. In some further embodiment, a method is provided for treating and/or preventing metabolic disorder or metabolic syndrome in a patient in need thereof, comprising administering to the patient a therapeutically effective amount of an ASK1 inhibitor (such as the compound of formula I, IA, or II, compounds 1, 2, or 3). In some embodiment, the metabolic disorder may be associated with type 2 diabetes mellitus. In certain embodiment, the metabolic disorder or syndrome may be modulated by fasting glucose, HbA1c, non-fasting glucose, improving insulin resistance, and/or reduced weight gain/weight loss. The modulation of certain glucose parameters (i.s. fasting glucose levels) may be determined using any suitable methods such as an oral glucose tolerance test.

In additional embodiment, provided herein is a method of treating, modulating, improving, or preventing weight loss or gain in a patient in need thereof, comprising administering to the patient a therapeutically effective amount of an ASK1 inhibitor, optionally in combination with a therapeutically effective amount of a LOXL2 inhibitor. In some embodiments, weight loss or gain may be associated with metabolic syndrome or type 2 diabetes mellitus.

In some embodiments, provided herein is a method for treating or preventing liver damage or injury in a patient in need thereof, comprising administering to the patient a therapeutically effective amount of an ASK1 inhibitor. In some other embodiments, the liver damage or injury may be acute or chronic. In certain embodiments, the acute liver damage or injury may be caused by alcoholic injury or drug overdosing. In certain other embodiment, the liver damage or injury is acetaminophen (APAP) hepatotocity. In other embodiments, the methods for treating or preventing acute liver damage or injury in a patient in need thereof compising administering to the patient a therapeutically effective amount of an ASK1 inhibitor. In some embodiment, the methods for treating or preventing acute liver alcoholic injury, drug overdosing, or APAP hepatotoxicity in a patient in need thereof, compising administering to the patient a therapeutically effective amount of an ASK1 inhibitor.

### ASK1 Inhibitors

An ASK1 inhibitor for use in the methods and pharmaceutical compositions disclosed herein may be any chemical compound or biological molecule (e.g., a protein or antibody) capable of inactivating apoptosis signal regulating kinase 1 (ASK1) protein. ASK1 inhibitors for use in the methods described herein are known (see, e.g., U.S. 2011/0009410 and U.S. Pat. No. 8,440,665, both of which are incorporated herein in their entirety) and/or can be identified via known methods (see, e.g., U.S. 2007/0276050 and U.S. 2011/0009410, which are incorporated herein by reference in their entirety).

In certain embodiments, the ASK1 inhibitor is a compound of formula (I):

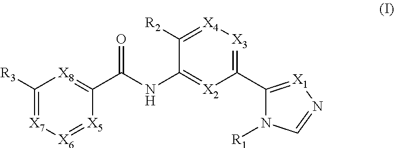

wherein:

R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, (C1-C15) alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or

R⁶ and R⁷ when taken together with the nitrogen to which they are attached form a heterocycle;

R² is hydrogen, halo, cyano, alkoxy, or alkyl optionally substituted by halo;

R³ is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;

X¹, X², X³, X⁴, X⁵, X⁶, X⁷ and X⁸ are independently C(R⁴) or N, in which each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, —O—R⁶; or

X⁵ and X⁶ or X⁶ and X⁷ are joined to provide optionally substituted fused aryl or optionally substituted fused heteroaryl; and

with the proviso that at least one of X², X³, and X⁴ is C(R⁴); at least two of X⁵, X⁶, X⁷, and X⁸ are C(R⁴); and at least one of X², X³, X⁴, X⁵, X⁶, X⁷ and X⁸ is N;

or a pharmaceutically acceptable salt, isomer, stereoisomer, or tautomer thereof.

In certain embodiments, the ASK1 inhibitor is a compound of formula (IA):

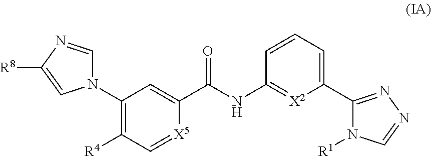

wherein:

R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)—R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, (C1-C15) alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or

R⁶ and R⁷ when taken together with the nitrogen to which they are attached form a heterocycle;

R⁸ is hydrogen, alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;

X² and X⁵ are independently C(R⁴) or N; and

each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, and —O—R⁶;

with the proviso that at least one of X² and X⁵ is N;

or a pharmaceutically acceptable salt, isomer, stereoisomer, or tautomer thereof.

Exemplary compounds of Formula (I) and (IA) for use in the methods and pharmaceutical compositions described herein can be found in Corkey et al. U.S. 2011/0009410, which is incorporated herein by reference in its entirety.

In certain embodiments, the ASK1 inhibitor is a compound of formula (II):

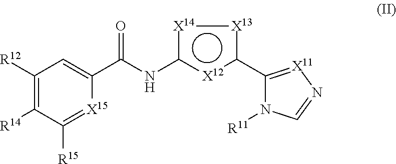

wherein:

R¹¹ is (C1-C6)alkyl, (C1-C6)alkenyl, (C1-C6)alkynyl, (C3-C6)cycloalkyl, aryl, heteroaryl, or heterocyclyl, wherein the (C1-C6)alkyl, (C1-C6)alkenyl, (C1-C6)alkynyl, (C3-C6)cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R¹⁶, C(O)R¹⁶, OC(O)R¹⁶C(O)OR¹⁶, C(O)N(R¹⁶)(R¹⁷), OC(O)N(R¹⁶)(R¹⁷), SR¹⁶, S(═O)R¹⁶, S(═O)₂R¹⁶, S(═O)₂N(R¹⁶)(R¹⁷), S(═O)₂OR¹⁶, N(R¹⁶)(R¹⁷), N(R¹⁶)C(O)R¹⁷, N(R⁶)C(O)OR¹⁷, N(R¹⁶)C(O)N(R¹⁶)(R¹⁷), N(R¹⁶)S(═O)₂R¹⁶, CN, and OR¹⁶, wherein the alkyl, cycloalkyl, heterocyclyl, aryl, and aryloxy are optionally substituted with from one to three substituents selected from alkyl, cycloalkyl, alkoxy, hydroxyl, and halo;

R¹⁶ and R¹⁷ are independently selected from the group consisting of hydrogen, (C1-C15)alkyl, cycloalkyl, heterocyclyl, aryl, and heteroaryl, wherein the (C1-C15)alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or

R¹⁶ and R¹⁷ when taken together with the nitrogen to which they are attached form a heterocycle;

R¹² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR¹⁶, OC(O)R¹⁶, OC(O)N(R¹⁶)(R¹⁷), SR¹⁶, N(R¹⁶)(R¹⁷), S(═O)R¹⁶, S(═O)₂R¹⁶, S(═O)₂N(R¹⁶)(R¹⁷), S(═O)₂OR¹⁶, N(R¹⁶)C(O)R¹⁷, N(R¹⁶)C(O)OR¹⁷, N(R¹⁶)C(O)N(R¹⁶)(R¹⁷), C(O)R¹⁶, C(O)OR¹⁶, C(O)N(R¹⁶)(R¹⁷), and N(R¹⁶)S(═O)₂R¹⁷, and wherein the alkyl, alkoxy, cycloalkyl, aryl, heteroaryl and heterocyclyl are optionally substituted with one or more substituents selected from halo, oxo, NO₂, alkyl, haloalkyl, haloalkoxy, N(R¹⁶)(R¹⁷), C(O)R¹⁶, C(O)OR¹⁶, C(O)N(R¹⁶)(R¹⁷), CN, OR¹⁶, cycloalkyl, aryl, heteroaryl and heterocyclyl; with the proviso that the heteroaryl or heterocyclyl moiety includes at least one ring nitrogen atom;

R¹⁴ and R¹⁵ are independently hydrogen, halo, cyano, (C1-C6)alkyl, (C1-C6)alkoxy, or (C1-C6)cycloalkyl, wherein the alkyl, alkoxy, and cycloalkyl are optionally substituted by halo or (C3-C8)cycloalkyl;

X¹¹ and X¹⁵ are independently C(R¹³) or N, wherein each R¹³ is independently hydrogen, halo, (C1-C6)alkyl, (C1-C6)alkoxy or (C3-C8)cycloalkyl, wherein the alkyl and cycloalkyl are optionally substituted with from one to five substituents selected from halo, oxo, CF₃, OCF₃, N(R¹⁶)(R¹⁷), C(O)R¹⁶, C(O)OR¹⁷, C(O)N(R¹⁶)(R¹⁷), CN, and OR¹⁶; and

X¹², X¹³ and X¹⁴ are independently C(R¹³), N, O, or S; with the proviso that at least one of X¹², X¹³, and X¹⁴ is C(R¹³); and only one of X¹², X¹³, and X¹⁴ is O or S;

or a pharmaceutically acceptable salt, isomer, stereoisomer, or tautomer thereof.

Exemplary compounds of Formula (II) for use in the methods and pharmaceutical compositions described herein can be found in Corkey et al. U.S. Pat. No. 8,440,665, which is incorporated herein by reference in its entirety. Additional exemplary ASK1 inhibitors, the methods of preparation thereof, or the methods of use thereof may be found in U.S. patent application publication nos. 2011/0009410 and US2013/0197037, each of which is incorporated herein by reference in the entirety.

In certain embodiments, the ASK1 inhibitor is:

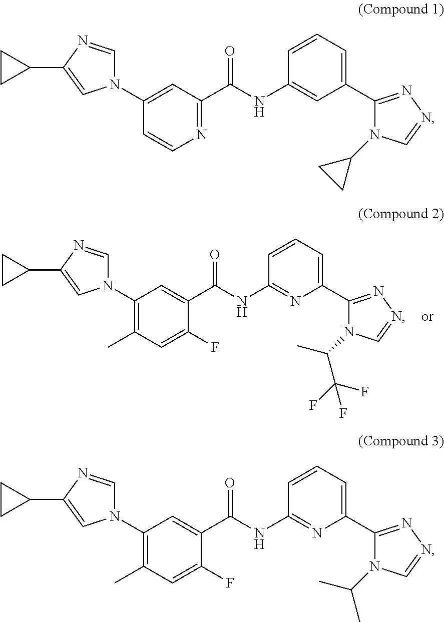 [SMILES: O=C(Nc1cccc(-c2nncn2C2CC2)c1)c1cc(-n2cnc(C3CC3)c2)ccn1 ; Cc1cc(F)c(C(=O)Nc2cccc(-c3nncn3[C@@H](C)C(F)(F)F)n2)cc1-n1cnc(C2CC2)c1 ; Cc1cc(F)c(C(=O)Nc2cccc(-c3nncn3C(C)C)n2)cc1-n1cnc(C2CC2)c1]

or a pharmaceutically acceptable salt thereof. Compound 1, Compound 2, and Compound 3 may be prepared according to U.S. patent publication nos. 2011/0009410 or US2013/0197037, each of which is incorporated herein by reference in the entirety.

The compounds described herein in which from 1 to n hydrogen atoms attached to a carbon atom may be replaced by a deuterium atom or D, in which n is the number of hydrogen atoms in the molecule. It is known that the deuterium atom is a non-radioactive isotope of the hydrogen atom. Such compounds may increase resistance to metabolism, and thus may be useful for increasing the half-life of the compounds described herein or pharmaceutically acceptable salts, isomers, prodrugs, or solvates thereof, when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium. In some embodiments, the compounds described herein may include the compounds having the structures of any of the formulae (I), (IA), (II), Compound 1, Compound 2, and Compound 3.

### LOXL2 Inhibitors

A LOXL2 inhibitor for use in the methods and pharmaceutical compositions described herein may be any agent that is capable of inactivating lysyl oxidase-like 2 (LOXL2) protein. The agent may be a chemical compound or biological molecule (e.g., a protein or antibody). Such inhibitors are readily identified by known methods (see, e.g., U.S. Pat. No. 8,461,303, U.S. 2009/0053224 and U.S. 2011/0044907, which are hereby incorporated herein by reference in their entirety).

In certain embodiments, the LOXL2 inhibitor is an anti-LOXL2 antibody (see, e.g., U.S. Pat. No. 8,461,303, U.S. 2012/0309020, U.S. 2013/0324705, and U.S. 2014/0079707, which are incorporated herein by reference in their entirety). The anti-LOXL2 antibody can be a monoclonal antibody (including full length monoclonal antibody), polyclonal antibody, human antibody, humanized antibody, chimeric antibody, diabody, multispecific antibody (e.g., bispecific antibody), or an antibody fragment including, but not limited to, a single chain binding polypeptide, so long as it exhibits the desired biological activity. Exemplified anti-LOXL2 antibody or antigen binding fragment thereof may be found in U.S. patent application publication nos. 2012/0309020, 2013/0324705, 2014/0079707, 2009/0104201, 2009/0053224, and 2011/0200606; each of which is incorporated herein by reference in the entirety.

In certain embodiments, the anti-LOXL2 antibody is a monoclonal anti-LOXL2 antibody, or antigen-binding fragment thereof. In other embodiments, the anti-LOXL2 antibody is a polyclonal anti-LOXL2 antibody, or antigen-binding fragment thereof. Such antibodies are known in the art or are available from commercial sources. In one embodiment, the anti-LOXL2 antibodies or antigen binding fragment thereof specifically binds to an epitope having an amino acid sequence set forth as SEQ ID NO: 1. In some embodiments, the anti-LOXL2 antibody is an isolated antibody or antigen binding fragment thereof, comprising the complementarity determining regions (CDRs), CDR1, CDR2, and CDR3, of a heavy chain variable region comprising the amino acid sequence set forth as SEQ ID NO: 2, 3, 4, or 5, and the CDRs, CDR1, CDR2, and CDR3, of a light chain variable region comprising the amino acid sequence set forth as SEQ ID NO: 6, 7, or 8, wherein the isolated antibody or antigen binding fragment thereof specifically binds a lysyl oxidase-like 2(LOXL2) protein. In other embodiments, CDR1, CDR2, and CDR3 of the heavy chain variable region comprise the amino acid sequences set forth as SEQ ID NOs: 9, 10, and 11, respectively, and the CDR1, CDR2, and CDR3 of the light chain variable region comprise the amino acid sequences set forth as SEQ ID NOs: 12, 13, and 14, respectively. In some other embodiments, the anti-LOXL2 antibody has a heavy chain variable region comprising the amino acid sequence set forth as SEQ ID NO: 2, 3, 4, or 5, and a light chain variable region comprising the amino acid sequence set forth as SEQ ID NO: 6, 7, or 8, wherein the isolated antibody or antigen binding fragment thereof specifically binds a lysyl oxidase-like 2 (LOXL2) protein. In further embodiment, the LOXL2 inhibitor is anti-LOXL2 antibody having the heavy chain variable region comprising the amino acid sequence set forth in SEQ ID NO: 4 and the light chain variable region comprising the amino acid sequence set forth in SEQ ID NO: 7. In further additional embodiments, the LOXL2 inhibitor is an anti-LOXL2 antibody comprising the sequences having about 85%, 90%, 91%, 92%, 93%, 94%, 95%, 96%, 97%, 98% or 99% identity to SEQ ID NO: 4. In some additional embodiments, the LOXL2 inhibitor is an anti-LOXL2 antibody comprising the sequences having about 85%, 90%, 91%, 92%, 93%, 94%, 95%, 96%, 97%, 98% or 99% identity to SEQ ID NO: 7. In certain embodiments, the isolated antibody or antigen binding fragment is humanized.

In additional embodiment, the LOXL2 inhibitor is anti-LOXL2 antibody AB0023 having the heavy chain variable region comprising the amino acid sequence set forth in SEQ ID NO: 15 and the light chain variable region comprising the amino acid sequence set forth in SEQ ID NO: 16. The methods of generating AB0023 and other anti-LOXL2 antibodies are generally disclosed in the '303 patent. In certain embodiments, the isolated antibody or antigen binding fragment is humanized. In further additional embodiments, the LOXL2 inhibitor is an anti-LOXL2 antibody comprising the sequences having about 85%, 90%, 91%, 92%, 93%, 94%, 95%, 96%, 97%, 98% or 99% identity to SEQ ID NO: 15. In some additional embodiments, the LOXL2 inhibitor is an anti-LOXL2 antibody comprising the sequences having about 85%, 90%, 91%, 92%, 93%, 94%, 95%, 96%, 97%, 98% or 99% identity to SEQ ID NO: 16. In other embodiment, the LOXL2 inhibitor is an anti-LOXL2 antibody having the CDRs of SEQ ID NO: 4 and the CDRs of SEQ ID NO: 7. In further embodiment, the LOXL2 inhibitor is an anti-LOXL2 antibody having the heavy chain variable region comprising the amino acid sequence set forth in SEQ ID NO: 4 and the light chain variable region comprising the amino acid sequence set forth in SEQ ID NO: 7. In additional embodiment, the LOXL2 inhibitor is anti-LOXL2 antibody AB0024 having the heavy chain variable region comprising the amino acid sequence set forth in SEQ ID NO: 4 and the light chain variable region comprising the amino acid sequence set forth in SEQ ID NO: 7. The methods of generating AB0024 and other anti-LOXL2 antibodies are generally disclosed in the '303 patent.

### Dosing and Administration

While it is possible for an active ingredient (i.e., the ASK1 inhibitor and/or the LOXL2 inhibitor) to be administered alone, it may be preferable to present them as pharmaceutical formulations or pharmaceutical compositions as described below. The formulations, both for veterinary and for human use, of the disclosure comprise at least one of the active ingredients (i.e., the ASK1 inhibitor and/or the LOXL2 inhibitor), together with one or more acceptable carriers therefor and optionally other therapeutic ingredients. The carrier(s) must be “acceptable” in the sense of being compatible with the other ingredients of the formulation and physiologically innocuous to the recipient thereof.

The active ingredients may be administered under fed conditions. The term “fed conditions” or variations thereof refers to the consumption or uptake of food, in either solid or liquid forms, or calories, in any suitable form, before or at the same time when the active ingredients are administered. For example, the active ingredients may be administered to the subject (e.g., a human) within minutes or hours of consuming calories (e.g., a meal). In some embodiments, the active ingredients may be administered to the subject (e.g., a human) within 5-10 minutes, about 30 minutes, or about 60 minutes of consuming calories.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

The therapeutically effective amount of active ingredient (i.e., the ASK1 inhibitor and/or LOXL2 inhibitor) can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. It is contemplated that the active ingredient may be administered once, twice or three times a day. Also, the active ingredient may be administered once or twice a week, once every two weeks, once every three weeks, once every four weeks, once every five weeks, or once every six weeks.

The therapeutically effective amount of active ingredient (i.e., the ASK1 inhibitor and/or LOXL2 inhibitor) can be readily determined by a skilled clinician using conventional dose escalation studies. In some embodiments, the ASK1 inhibitor (including Compound 1, Compound 2, and Compound 3), the composition or the formulation thereof, will be administered in a dose from about 0.01 milligrams (mg) to 2 grams (g), about 0.1 mg to 450 mg, about 0.5 mg to about 250 mg, about 0.5 mg to 100 mg, about 0.5 mg to 50 mg, about 0.5 mg to 40 mg, about 0.5 mg to 30 mg, about 0.5 mg to 20 mg, about 0.5 mg to 10 mg, about 0.5 mg to 5 mg, about 0.5 mg to 4 mg, about 0.5 mg to 3 mg, about 0.5 mg to 2 mg, about 0.5 mg to 1 mg, about 1 mg to 250 mg, about 1 mg to 100 mg, about 1 mg to 50 mg, about 1 mg to 40 mg, about 1 to 35 mg, about 1 mg to 30 mg, about 1 to 25 mg, about 1 mg to 20 mg, about 1 to 15 mg, about 1 mg to 10 mg, about 1 mg to 5 mg, about 1 mg to 4 mg, about 1 mg to 3 mg, or about 1 mg to 2 mg. In another embodiment, the dosage ranges from about 1 mg or 100 mg. In some other embodiment, the dosage ranges from about 1 mg to 30 mg. In certain other embodiment, the dosage ranges from about 1 mg to 20 mg. In one embodiment, the dosage is about 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 mg. It is contemplated that the active ingredient, the composition or the formulation thereof, may be administered once, twice, or three times a day. Also, the active ingredient, the composition or the formulation thereof, may be administered once or twice a week, once every two weeks, once every three weeks, once every four weeks, once every five weeks, or once every six weeks. In other embodiment, the ASK1 inhibitor (i.e. Compound 1, Compound 2, and Compound 3), the composition or the formulation thereof, is administered once daily at the dose of 1, 2, 6, 10, 18, 20, 30, or 100 mg. In additional embodiment, the ASK1 inhibitor (i.e. Compound 1, Compound 2, and Compound 3), the composition or the formulation thereof, is presented in a tablet at a dose unit of 1, 2, 6, 10, 18, and 100 milligrams (mg) and the tablets contain pharmaceutically acceptable excipients. In another embodiment, the ASK1 inhibitor (i.e. Compound 1, Compound 2, and Compound 3), the composition or the formulation thereof, is administered orally once daily at the dose of 6 mg. In some another embodiment, the ASK1 inhibitor (i.e. Compound 1, Compound 2, and Compound 3), the composition or the formulation thereof, is administered orally once daily at the dose of 18 mg.

In certain embodiments, the LOXL2 inhibitor is an antibody that binds LOXL2 or antigen binding fragment thereof (including AB0023 and AB0024). In certain other embodiment, the anti-LOXL2 antibody or antigen binding fragment thereof, the composition or the formulation thereof, is administered at between about 25 mg to about 800 mg per subject. In some embodiments, the dosage is about 50 mg, about 100 mg, at about 150 m, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg per subject, including any range in between these values. In some embodiments, the anti-LOXL2 antibody or the antigen binding fragment thereof, the composition or the formulation thereof, of the above dosage is administered once a week, once every two weeks, once every three weeks, once a month, once every two months, once every three months, or once every six months. In one embodiment, the anti-LOXL2 antibody or antigen binding fragment thereof, the composition or the formulation thereof is delivered by intravenous administration (which may be referred to as intravenous infusion) or subcutaneous administration (which may be referred to as subcutaneous injection). In some embodiments, the anti-LOXL2 antibody or antigen binding fragment thereof, the composition or the formulation thereof, is administered subcutaneously at about 75 mg or 125 mg once a week. In certain embodiment, the anti-LOXL2 antibody or antigen binding fragment thereof, the composition or the formulation thereof, is administered intravenously at about 200 mg or 700 mg once a month. In additional embodiment, the anti-LOXL2 antibody or antigen-binding fragment thereof, the composition or the formulation thereof is administered subcutaneously (i.e. subcutaneous injection) at about 75 mg once a week. In one embodiment, the anti-LOXL2 antibody or antigen-binding fragment thereof, the composition or the formulation thereof is administered subcutaneously at about 125 mg once a week.

In additional embodiment, the ASK1 inhibitor (i.e. Compound 1, Compound 2, and Compound 3) the composition or the formulation thereof, which is administered orally once daily at the dose of 6 mg, may be optionally combined (i.e. administered simultaneously or sequentially) with the anti-LOXL2 inhibitor (i.e. AB0023 and AB0024), which is administered subcutaneously once a week at the dose of 75 mg. In certain additional embodiment, the ASK1 inhibitor (i.e. Compound 1, Compound 2, and Compound 3) the composition or the formulation thereof, which is administered orally once daily at the dose of 6 mg, may be optionally combined (i.e. administered simultaneously or sequentially) with the anti-LOXL2 inhibitor (i.e. AB0023 and AB0024) which is administered subcutaneously once a week at the dose of 125 mg. In some additional embodiment, the ASK1 inhibitor (i.e. Compound 1, Compound 2, and Compound 3) the composition or the formulation thereof, which is administered orally once daily at the dose of 18 mg, is combined (i.e. administered simultaneously or sequentially) with the anti-LOXL2 inhibitor (i.e. AB0023 and AB0024) which is administered subcutaneously once a week at the dose of 75 mg. In further additional embodiment, the ASK1 inhibitor (i.e. Compound 1, Compound 2, and Compound 3) the composition or the formulation thereof, which is administered orally once daily at the dose of 18 mg, may be optionally combined (i.e. administered simultaneously or sequentially) with the anti-LOXL2 inhibitor (i.e. AB0023 and AB0024) which is administered subcutaneously once a week at the dose of 125 mg.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In certain cases, the ASK1 inhibitor, with or without a LOXL2 inhibitor, is administered with food. In one embodiment, the patient is human.

When used in combination in the methods disclosed herein, the ASK1 inhibitor and the LOXL2 inhibitor can be administered together in a single pharmaceutical composition, or serperatly (either concurrently or sequentially) in more than one pharmaceutical composition. In certain embodiments, the ASK1 inhibitor and the LOXL2 inhibitor are administered together. In other embodiments, the ASK1 inhibitor and the LOXL2 inhibitor are administered separately. In some aspects, the ASK1 inhibitor is administered prior to the LOXL2 inhibitor. In some aspects, the LOXL2 inhibitor is administered prior to the ASK1 inhibitor. When administered separately, the ASK1 inhibitor and the LOXL2 inhibitor can be administered to the patient by the same or different routes of delivery. For example, the ASK1 inhibitor may be administered orally and the LOXL2 inhibitor may be administered subcutaneously.

### Pharmaceutical Compositions

The pharmaceutical compositions of the disclosure provide for an effective amount of an ASK1 inhibitor, with or without, an effective amount of a LOXL2 inhibitor.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material (i.e., an ASK1 inhibitor, a LOXL2 inhibitor, or combination thereof) compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In embodiments where the ASK1 inhibitor is administered in combination with a LOXL2 inhibitor, the ASK1 inhibitor and LOXL2 inhibitor may be administered together in a combination formulation or in separate pharmaceutical compositions, where each inhibitor may be formulated in any suitable dosage form. In certain embodiments, the methods provided herein comprise administering separately a pharmaceutical composition comprising an ASK1 inhibitor and a pharmaceutically acceptable carrier or excipient and a pharmaceutical composition comprising a LOXL2 inhibitor and a pharmaceutically acceptable carrier or excipient. Combination formulations according to the present disclosure comprise an ASK1 inhibitor and a LOXL2 inhibitor together with one or more pharmaceutically acceptable carriers or excipients and optionally other therapeutic agents. Combination formulations containing the active ingredient (i.e. an ASK1 inhibitor and a LOXL2 inhibitor) may be in any form suitable for the intended method of administration.

It is understood that the below examples illustrate certain aspects of the present application. It is also understood that values and parameters shown in the examples may be modified within reasonable variation, and that various modifications may be made within the scope of the present application.

### EXAMPLES

The following abbreviations used herein have the following meanings.

| | |
|---|---|
| ALT | Alanine aminotransferase |
| ASK1 | Apoptosis signal-regulating kinase 1 |
| AST | Aspartate aminotransferase |
| AUC | Area under the curve |
| CDRs | Complementarity determining regions |
| dL | Deciliter |
| EC₅₀ | Half maximal effective concentration |
| EDTA | Ethylenediaminetetraacetic acid |
| ELISA | Enzyme-linked immunosorbent assay |
| FFPE | Formaldehyde Fixed-Paraffin Embedded |
| g | Grams |
| hr/hrs | Hour/hours |
| HSCs | hepatic stellate cells |
| HYP | Hydroxyproline |
| IU | International units |
| L | Liter |
| LOXL2 | lysyl oxidase-like 2 |
| M | Molar |
| min | Minute |
| mg | Milligram |
| mL | Milliliter |
| NAFLD | Non-alcoholic fatty liver disease |
| NASH | Nonalcoholic steatohepatitis |
| ng | Nanograms |
| nM | Nanomolar |
| OGTT | Oral glucose tolerance test |
| PBC | Primary biliary cirrhosis |
| PCA | Percent collagen area |
| PFA | Paraformaldehyde |
| pg | Picograms |
| PSC | Primary sclerosing cholangitis |
| RNA | Ribonucleic acid |
| ROS | reactive oxygen species |
| rpm | Revolutions per minute |
| TGF-β | Transforming growth factor beta |
| α-SMA | α-smooth muscle actin |
| μg | Microgram |

### Example 1

### Effect of ASK1 Inhibitors on Human Hepatic Stellate Cells

In this example, the activities of Compounds 1, 2, and 3 were evaluated in primary human HSCs (Sciencell). The structures of Compounds 1, 2, and 3 are shown below:

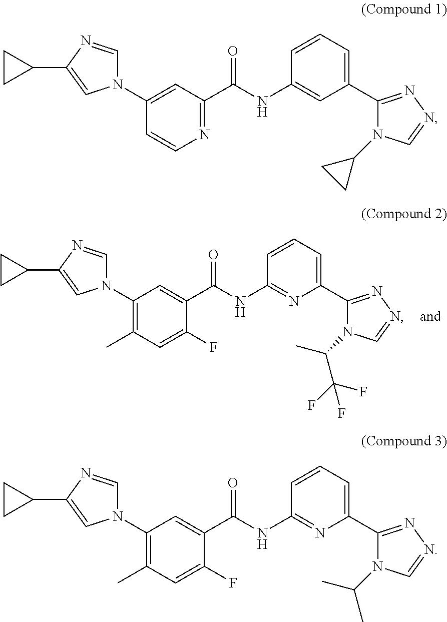 [SMILES: O=C(Nc1cccc(-c2nncn2C2CC2)c1)c1cc(-n2cnc(C3CC3)c2)ccn1 ; Cc1cc(F)c(C(=O)Nc2cccc(-c3nncn3[C@@H](C)C(F)(F)F)n2)cc1-n1cnc(C2CC2)c1 ; Cc1cc(F)c(C(=O)Nc2cccc(-c3nncn3C(C)C)n2)cc1-n1cnc(C2CC2)c1]

TGF-β and ROS-mediated activation of hepatic stellate cells (HSCs) has been shown to increase collagen synthesis and α-SMA expression. Collagen synthesis, α-SMA (smooth muscle actin) expression, and changes in transcript expression were measured in response to stimulation from ROS (reactive oxygen species) and TGF-β.

Collagen Assay:

Commercially available primary human HSCs (Sciencell) were plated at a density of 250,000 cells/well on 12-well plates (Corning). HSCs were co-stimulated with 5 ng/mL TGF-β+/− ASK1 inhibitor compounds. Collagen production was assessed by Sircol Assay where extracellular collagen was digested with pepsin, followed by acid extraction, neutralization, and colometric quantification using the Sircol assay kit according to the manufacturer's protocols (BioColor).

α-SMA Assay:

For quantification of α-SMA protein expression, HSC's were plated at a density of 10,000 cells/well in collagen coated 96-well plates (BD-Biosystems). HSCs were co-stimulated with 5 ng/mL TGF-β (R&D systems) with ASK1 inhibitor compounds (Compound 1, Compound 2 and Compound 3, shown below) for 24 hrs. HSCs were fixed in 4% PFA, permeabilized in 0.02% Triton-X-100, blocked for 30 min. and stained with an anti-α-SMA specific antibody (Sigma) and a flourescently labeled secondary antibody (Invitrogen). α-SMA expression was quantified on a fluorescent plate reader.

RT-PCR Analysis:

For gene expression assays, RNA was isolated using commercially available kit (Qiagen Midikit), converted to cDNA (Retroscript Kit, Ambion) and tested for expression of genes involved in fibrosis with human probes on ABI 7300 (Applied Biosystems). All assays were performed in triplicate. Positive and negative controls were included to help with the interpretation of results.

The data in Table 1 shows the activities of the test compounds in the above-described assays.

TABLE 1

| | Collagen Assay | α-SMA Assay |
|---|---|---|
| ASK1 Inhibitor | EC₅₀ (nM) | EC₅₀ (nM) |
| Compound 1 | 22.4 | 62.4 |
| Compound 2 | 8.0 | 72.9 |
| Compound 3 | 11.6 | 18.9 |

Additional assays were conducted with HSCs which were pre-treated with Compounds 2 and 3 then stimulated with TGF-β1 (5 ng/ml for 48 hrs at 37° C.). Quantitative RT-PCR was used to determine the effects of compounds to inhibit TGF-β1-induced gene transcription of αSMA, COL1α1, metalloproteinase-1 (TIMP1), and lumican. Five ng/mL of TGF-β1 was shown to induce a 4.8±2.6 and 3.7±0.1 fold induction of metalloproteinase-1 (TIMP1) and osteopontin (OPN), respectively.

The results showed that both Compounds 2 and 3 blocked or inhibited TGF-β1-induced responses in RNA levels of αSMA, COL1α1, TIMP1, and lumican in HSCs. The EC₅₀ values for Compound 3 were 26.4 nM, 7.9 nM, 1.9 nM, and 12.8 nM for inhibiting TGF-β1-induced production of collagen, αSMA, TIMP1, and OPN, respectively. Taken together, the results showed that ASK1 inhibitors, such as Compounds 1-3, inhibit TGF-β1 signaling which are involved in fibrogenesis and may be a potential anti-fibrotic agent for treating patients having NASH.

### Example 2

### Effect of ASK1 Inhibitor in the Models of NASH or PSC

Oxidative stress pathways are implicated in the pathogenesis of NASH. Reactive oxygen species (ROS) drive hepatic stellate cell (HSCs) activation by increasing collagen production and α-SMA expression. Apoptosis signal-regulating kinase 1 (ASK1) responds to ROS by regulating the p38 and JNK pathways. Activation of the ASK1 pathway in human NASH patients was evaluated using a selective small molecule ASK1 inhibitor in murine models of NASH and PSC.

Methods:

High fat, high cholesterol, high sugar diet was administered continuously to 12-week old male C57BL/6 mice concomitantly with vehicle or a selective small molecule ASK1 inhibitor, Compound 1. Compound 1 was mixed in a commercially available Western diet (Research Diets, Catalogue# D12079B) and administered continuously in 10-12 week old male C57BL/6 mice. Western diet includes 20 wt % protein, 50 wt % carbohydrate and 21 wt % fat, and is comprised of casein (195 g, 80 mesh), L-cystine (3 g), corn Starch (50 g), maltodextrin 10 (100 g), sucrose (341 g), cellulose (50 g), milk fat, anhydrous (200 g), corn oil (10 g), mineral mix 510001 (35 g), calcium carbonate (4 g), vitamin mix V10001 (10 g), choline bitartrate (2 g), cholesterol, USP (1.5 g) and ethoxyquin (0.04 g). Anhydrous milk fat typically contains approximately 0.3% cholesterol, and on this basis, the Western diet administered to the subjects contains approximately 0.21% cholesterol.

Animals were administered with high fructose corn syrup in drinking water (˜250 mL per animal per week starting on Day 1) and singly housed in standard-sized cages. Drinking water was made up of 23.1 g fructose (Sigma, Catalog # F2543) and 17.2 g glucose (Sigma, Catalog #49158) in 1000 mL of drinking water (Greenfield city water). Body weight, food and water consumption were measured weekly. Nonfasting blood glucose levels were measured monthly. The study was completed at 90 day intervals (Day 90, 180, and 270).

RNA transcripts were evaluated in NASH (n=16) and healthy (n=8) human liver biopsies by qRT-PCR. Results are shown in Example 5 described below.

Oral glucose tolerance tests were conducted 1 week prior to sacrifice. Clinical chemistry (AST, ALT, cholesterol, and triglycerides) were measured at Covance Central Laboratory (Greenfield, Ind.). Hepatic steatosis grade and fibrosis severity (F-score) were assessed by a veterinary pathologist blinded to the treatment conditions. The Brunt staging system was utilized to score liver disease severity. Analysis was performed on FFPE (formalin fixed paraffin embedded) tissue from the right lateral and left medial lobes of the liver. Quantitative morphometric measurement of Picrosirius Red staining was performed on FFPE tissue from the right lateral and left medial lobes using StainMap (Flagship Biosciences). Liver was collected (100-200 mg) during necropsy and snap frozen in liquid N₂ for hydroxyproline (HYP) ELISA for determination of collagen content (Quickzyme Biosciences).

Murine NASH Model:

At days 90, 180, 270, and 360, cohorts of animals (n=10/time/treatment group) were evaluated for metabolic parameters and fibrosis endpoints including liver hydroxyproline (HYP) levels and liver histology.

Murine Mdr2^{-/-} PSC Model:

At 4 weeks of age, animals (n=20/group) were treated with either vehicle or Compound 1 for 4 and 8 weeks. Liver fibrosis was assessed by HYP and liver histology. Histological staging was performed by a veterinary pathologist blinded to the treatment conditions.

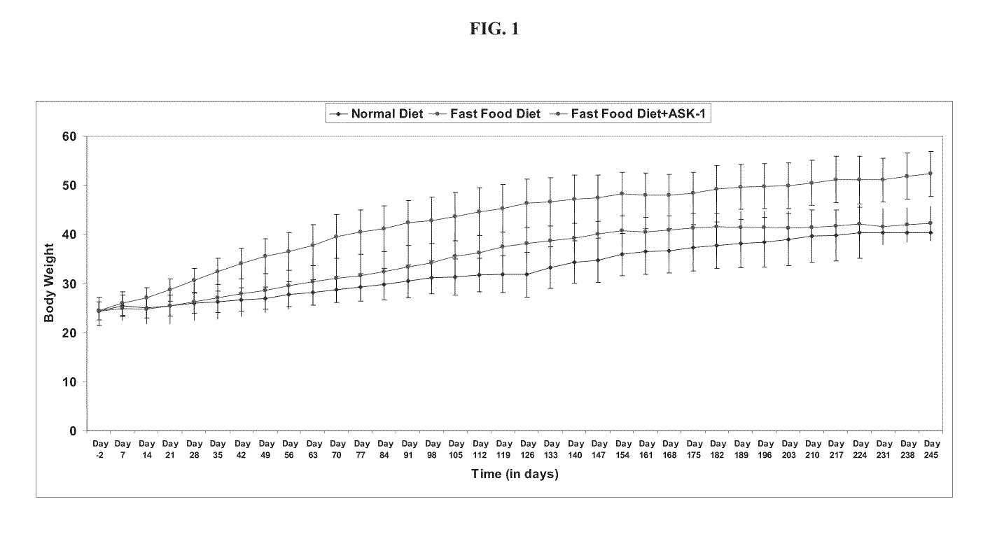

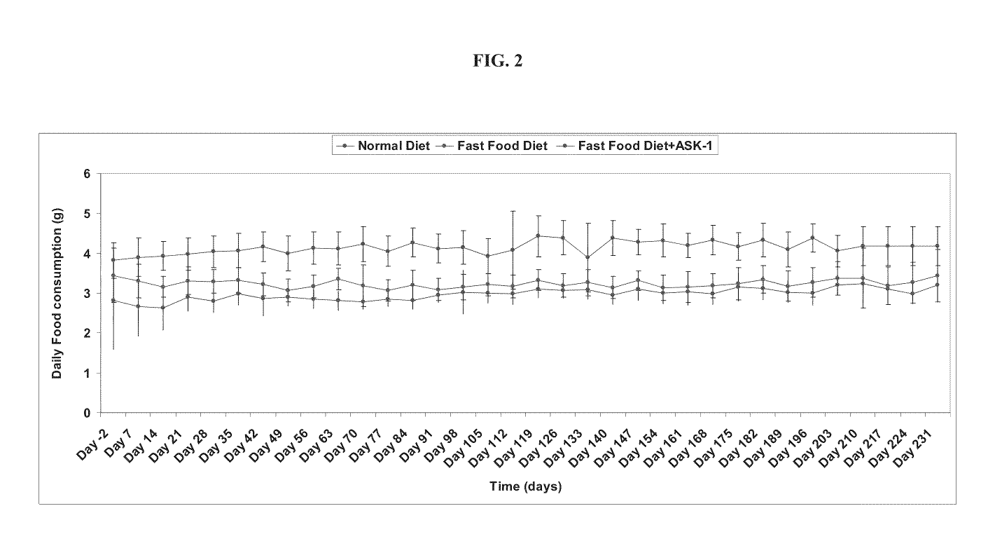

ASK1-treated mice gained less weight than high fat diet fed (untreated) animals over 270 day treatment duration, despite consuming comparable amounts of food and drinking water (FIG. 1 and FIG. 2). At Day 270, both body weight and food consumption were consistent with earlier time points.

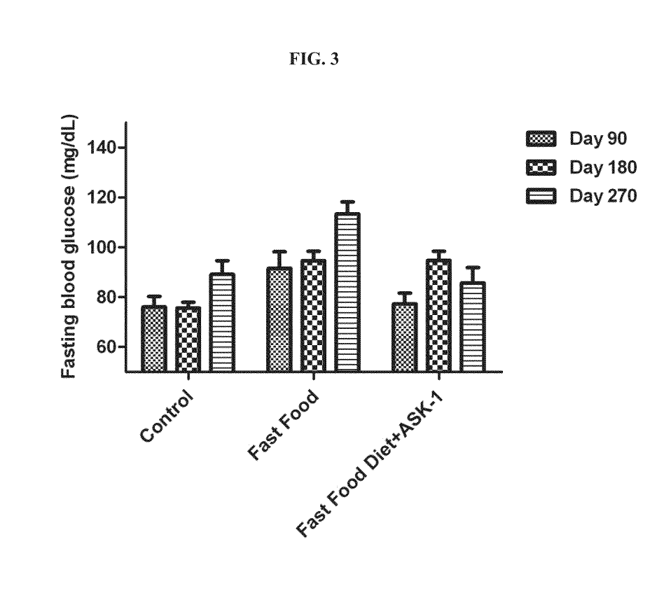

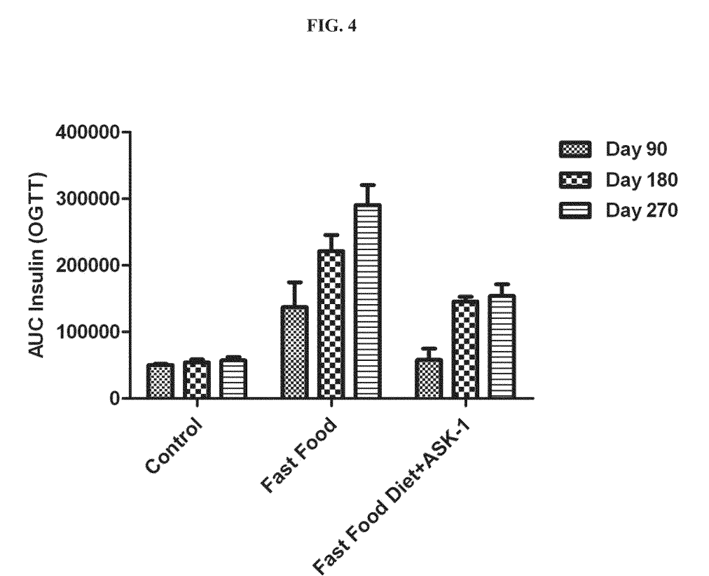

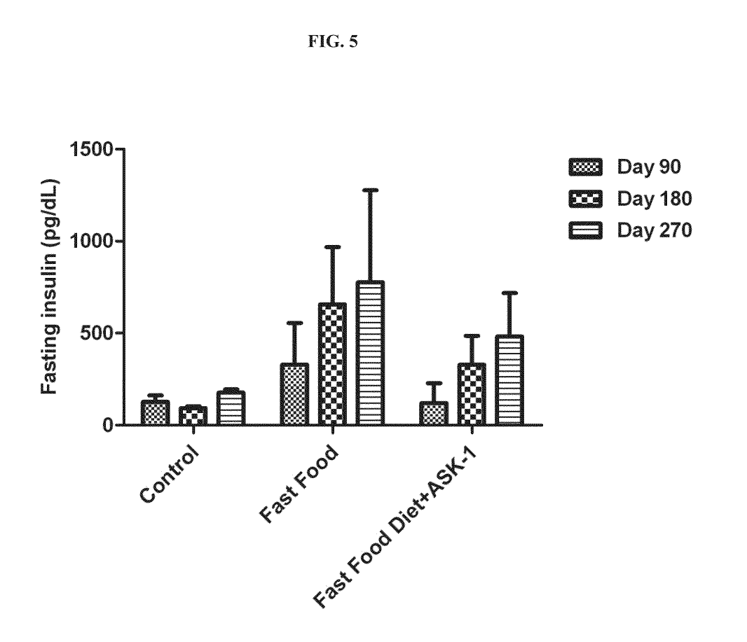

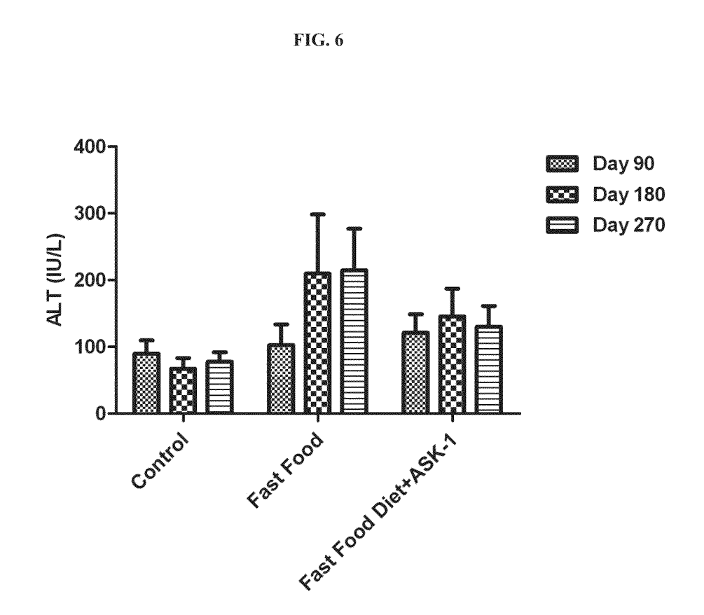

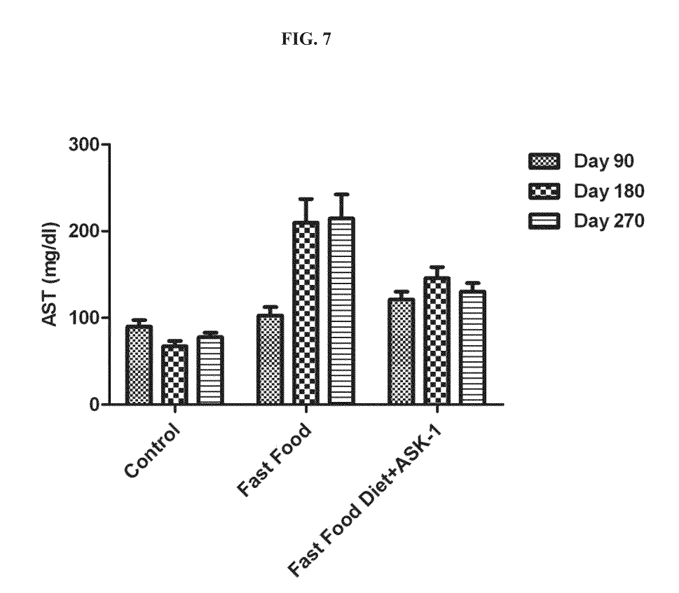

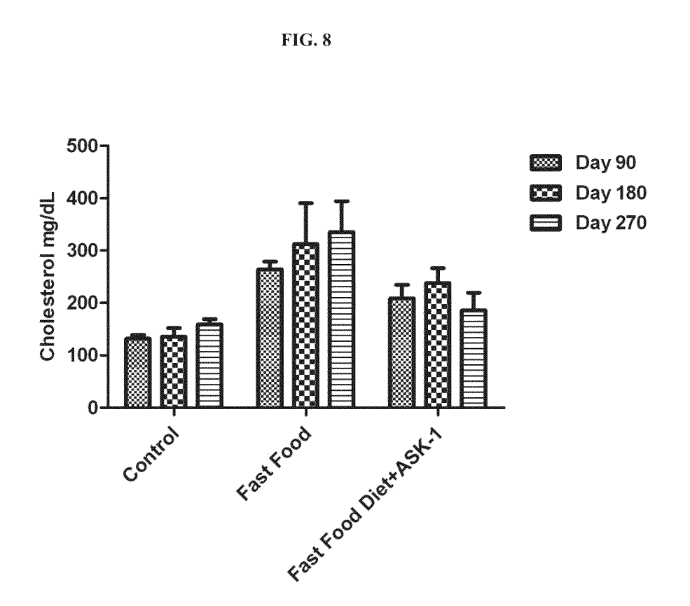

At 270 days, no statistically significant differences were observed in nonfasting blood glucose levels and in AUC glucose during an oral glucose tolerance test (OGTT). However, animals treated with Compound 1 exhibited lower blood glucose levels than control animals not treated with Compound 1 (FIG. 3). Also, mice treated with Compound 1 exhibited statistically significant lower AUC insulin levels (FIG. 4) and fasting insulin levels (FIG. 5) during an oral glucose tolerance test. In addition, mice treated with Compound 1 showed a reduction in liver function enzymes, ALT (FIG. 6) and AST (FIG. 7), and serum cholesterol levels (FIG. 8).

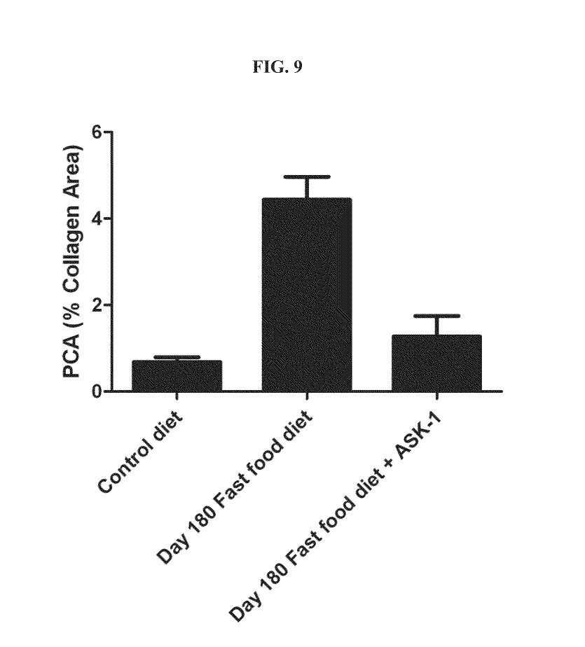

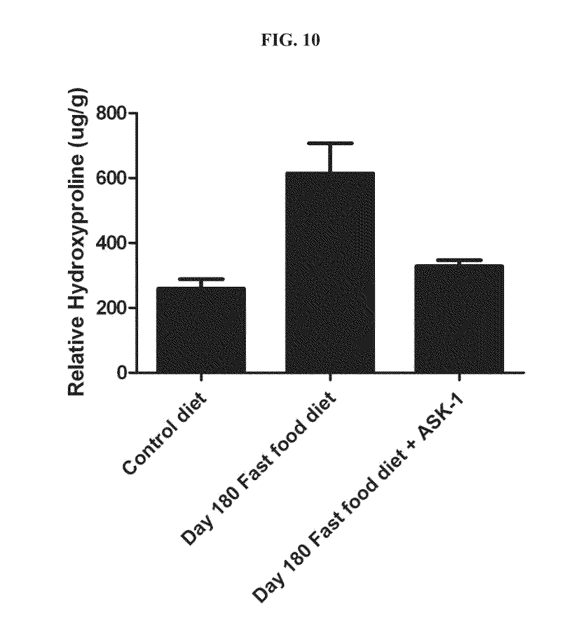

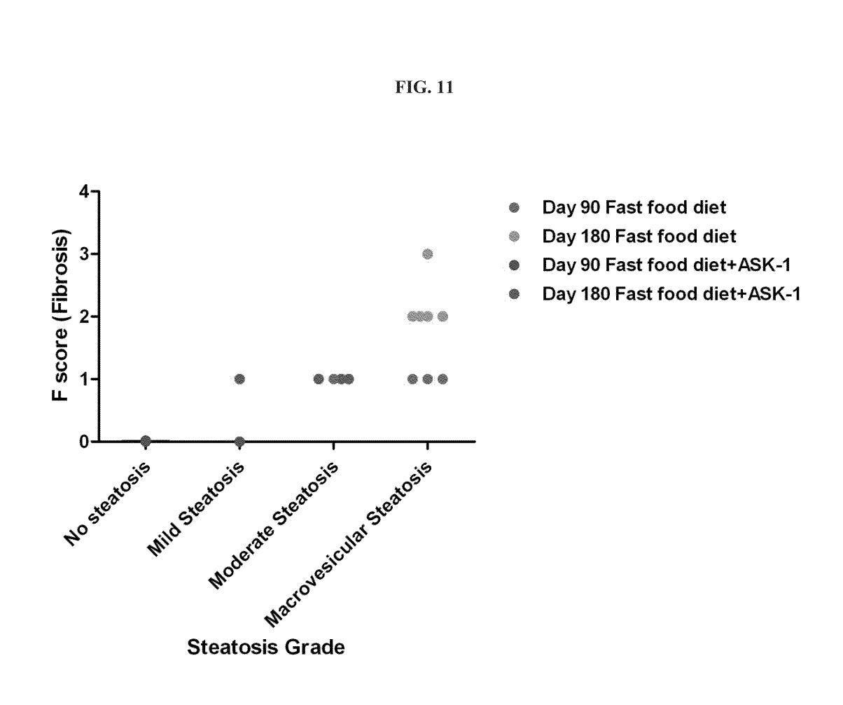

At 180 days, liver histological assessment revealed a 71% reduction in percent collagen area (PCA) by quantitative morphometry (FIG. 9). Data from the hydroxyproline assay corroborates these results with a 52% reduction in liver collagen content (FIG. 10). Hepatic steatosis grade and fibrosis severity (F-score) was assessed at 90 and 180 days. The data shows that, at both 90 and 180 days, the ASK1 treated mice had a lower steatosis grade and a lower F-score than the untreated animals at the same day (FIG. 11).

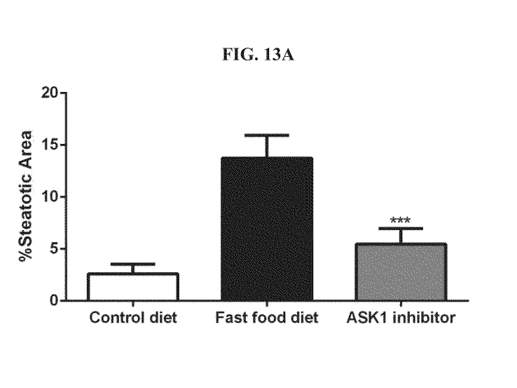

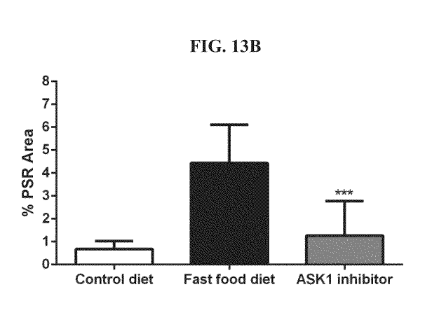

As shown in FIGS. 13A and 13B, the Compound 1 treatment blocked or prevented steatosis and fibrosis progession. In this experiment, whole slide-scan images of Hematoxylin & Eosin (H&E) and Picrosirius Red (PSR) stained slides were captured using a Leica SCN400 scanner at 40× magnification. Quantitative image analysis was performed on the whole slide-scan images using Definiens Tissue Studio Architect XD (Definiens Inc.) to determine the extent and intensity of either steatosis or PSR staining. For the steatosis measurement, H&E stained slides were used. The contrast between white, lipid-laden hepatocytes and the surrounding pink and blue parenchyma was exploited to quantify the percentage of steatotic cells. The cells in the “negative” category were found to be associated with cells determined to be steatotic by visual examination. The percentage of cells in the “negative” category was utilized to express the relative number of steatotic cells within the liver sections examined. PSR staining was quantified by measuring the total PSR-stained area and was expressed as a percentage of total liver parenchymal area. The results were summarized in FIG. 13. At 180 days, the level of hepatic steatosis of the mice treated with Compound 1 was reduced from 13.7% to 5.5%, a 59.8% reduction as determined by histology. Also, the percent area staining positive for picrosirius red of the mice treated with Compound 1 was reduced from 4.4% to 1.3%, a 70% reduction.

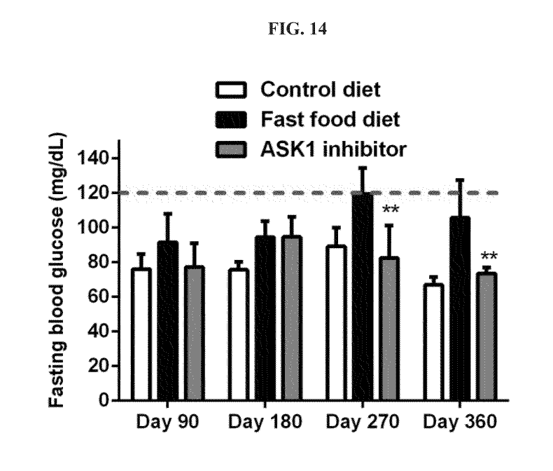

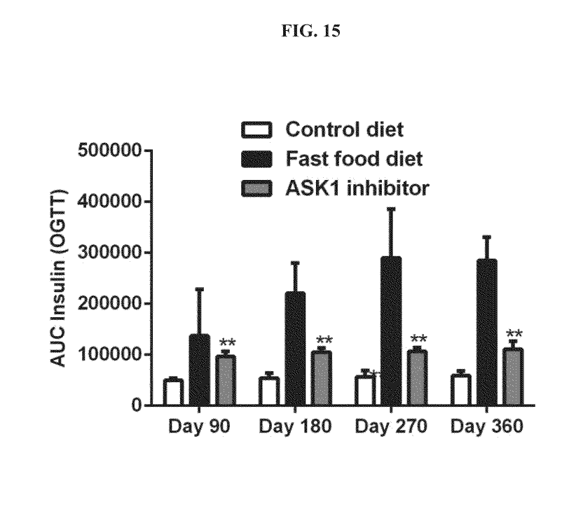

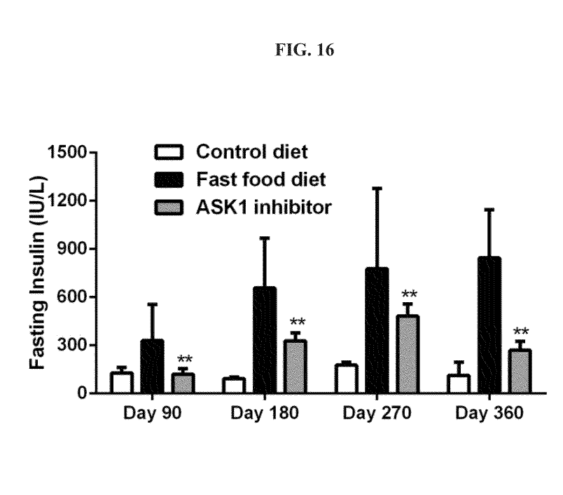

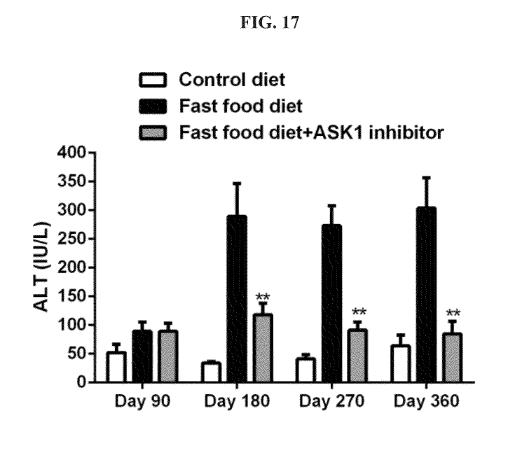

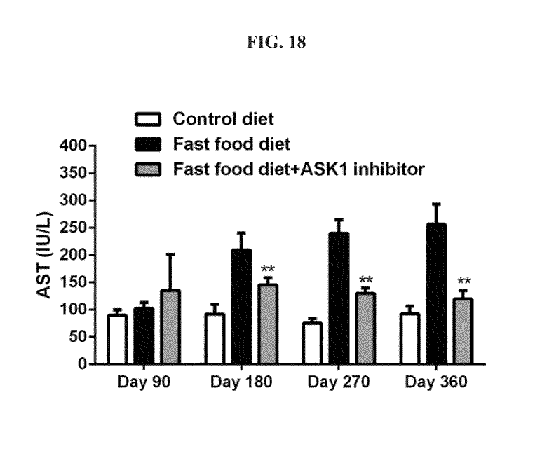

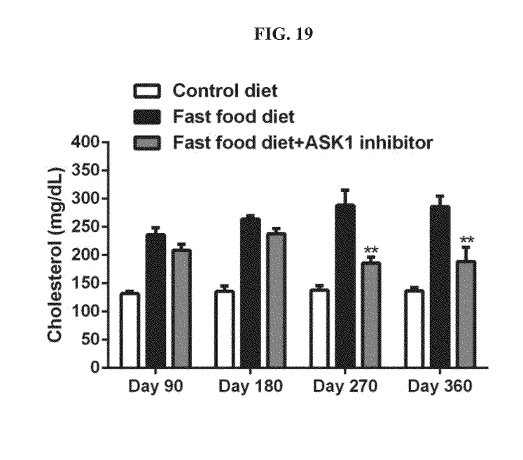

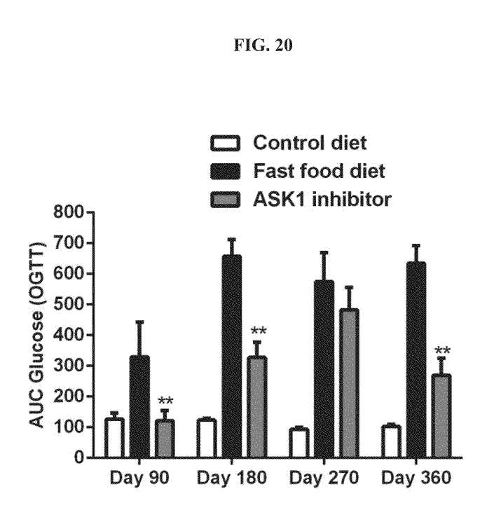

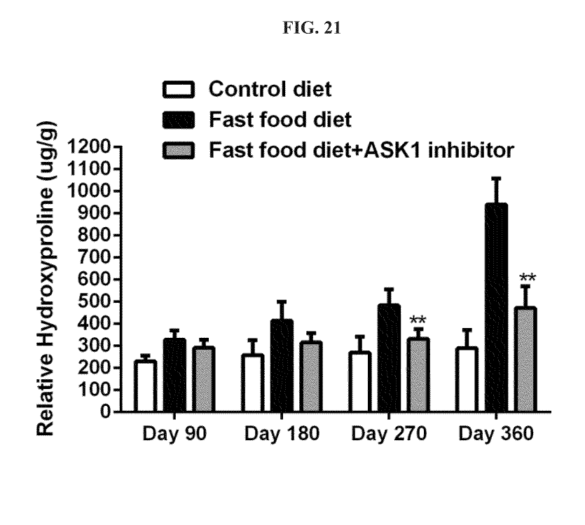

At 360 days, the fasting blood glucose (FIG. 14), AUC insulin (FIG. 15), fasting insulin (FIG. 16), ALT (FIG. 17), AST (FIG. 18), cholesterol (FIG. 19), AUC glucose (FIG. 20), and relative hydroxyproline (FIG. 21) levels were tested in the different groups. FIGS. 14-21 show the analyses at day 90 (i.e. 90 days after treatment), 180, 270, and 360. On day 360, the levels of ALT (FIG. 17), AST (FIG. 18), and cholesterol (FIG. 19) in the mice treated with Compound 1 exhibited 72%, 53%, and 34% reduction, respectively, compared with those of the fast food diet mice. The Compound 1 treated mice had reduction in insulin resistance as shown by a 72% reduction in fasting insulin levels on day 360 (FIG. 16) and improvement in glucose metabolism (FIG. 14). This suggests that ASK1 inhibitor treatment blocked or prevent fibrosis progression in murine NASH model. Hepatic HYP levels were 940 pg/g at day 360 in the fast food diet mice and 471 μg/g in the mice treated with Compound 1, a 49% reduction (FIG. 21).

The animals treated with ASK1 inhibitor were resistant to diet-induced body weight gain, had an improved lipid profile, and reduced AST, ALT, and M30 (a marker of hepatocyte apoptosis) levels. Also, the animal treated with ASK1 inhibitor had a 51% reduction in fasting insulin levels, and 17% and 13% improvement in glucose and insulin AUC during oral glucose challenge.

In NASH liver biopsies, ASK1 pathway activation was increased based on decreased expression of Trx, an ASK1 inhibitor, and increased expression of TxNIP, an inducer of ASK1 signaling. Increased TxNIP levels were associated with higher TGF-β1, αSMA, and Col1a1 expression.

In a murine NASH model, treatment with the ASK1 inhibitor reduced hepatic steatosis, inhibited fibrosis progression, and reduced αSMA, p-P38, and collagen expression.

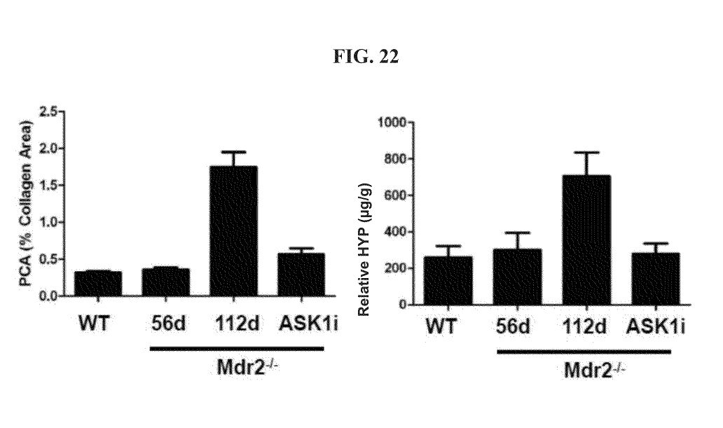

In a murine Mdr2^{-/-} PSC model, treatment with ASK1 inhibitor reduced fibrosis progression by 15% and 30% after 4 and 8 weeks, respectively and markers of fibrosis (P-IIINP, HA, TIMP-1) were significantly reduced. The percentage of collagen area and relative hydroxyproline in the mice treated with Compound 1 for 56 and 112 days were shown in FIG. 22.

The ASK1 signaling pathway in liver is active in human NASH. Inhibition of ASK1 prevented progression of hepatic fibrosis and steatosis and improved metabolic parameters in a NASH model. ASK1 inhibition also reduced fibrosis in an Mdr2^{-/-} PSC model. This data suggests that ASK1 was involved in the pathogenesis and progression of NASH and PSC. As shown in the study, an ASK1 inhibitor treatment prevented disease progression in a murine NASH model and blocked hepatic fibrosis in a murine PSC model. These results suggest that an ASK1 inhibitor may provide prophylactic effects in treating NASH and PSC.

### Example 3

### Effects of LOXL2 Inhibitor and ASK1 Inhibitor in the Model of NASH or PSC

Compound 1, an ASK1 inhibitor, blocked TAA-induced fibrosis progression in vivo and TGF-β signaling in hepatic stellate cells. Lysyl oxidase like-2 (LOXL2) enzymatically crosslinks collagen and is highly expressed during fibrogenesis. A murine antibody AB0023 directed against LOXL2 blocked fibrosis progression in TAA-induced liver fibrosis and in Mdr2^{-/-} mice.

Aliquots of snap-frozen liver (200 mg) were incubated for 16 hrs in 3 mL of 6 M HCl at 116° C. Liver homogenate (1.5 mL) was transferred to 1.5 mL Eppindorf tubes and centrifuged at 14,000 rpm for 10 min. Supernate was transferred to 96-deep well plate and diluted 1:1 with 4 M HCl. HYP levels were quantified using a commercially available hydroxyproline assay (Quickzyme Biosciences).

Interventional NASH Model:

Commercially available high fat, high cholesterol, high sugar diet was administered continuously to 12 week old male C57BL/6 mice for 320 days. At day 240, animals were administered Compound 1 (administered by weight as admixture in chow at 0.15%), AB0023 (30 mg/kg twice weekly, I.P.) or Compound 1 in combination with AB0023. Animal cohorts (n=15) were sacrificed after 80 days of treatment. Collagen synthesis and hydroxyproline (HYP) were measured using D₂O labeling. Second harmonic imaging was used to quantitate collagen morphometry.

Interventional PSC Model:

12-week old Mdr2^{-/-} were treated with Compound 1, AB0023, or Compound 1 in combination with AB0023 (Compound 1+AB0023). Cohorts (n=20) of animals were sacrificed after 56 days of treatment and evaluated for liver histology and tissue collagen content. The Mdr2(abcb4)−/− mice on the fibrosis-susceptible BALB/cAnNCrl background, develop spontaneous biliary fibrosis with features of primary sclerosing cholangitis, were generated (Ikenaga et al., A New Mdr2 Mouse Model of Sclerosing Cholangitis with Rapid Fibrosis Progression, Early-Onset Portal Hypertension, and Liver Cancer. American Journal of pathology 2014). This model exhibited accelerated progression of hepatic fibrosis to cirrhosis, early-onset portal hypertension, liver cancer, and was used to investigate human PSC treatments. At 6 weeks of age, the mice were administered with Compound 1 (0.15% in diet), AB0023 (30 mg/k/week ip), or the combination of Compound 1 (0.15% in diet) and AB0023 (30 mg/k/week i/p) (n=9-11/group) for 6 weeks. Control groups received treatment with either vehicle or an unrelated isotype-matched control IgG (i.e. a murine IgG1). Portal venous pressure

(PVP) was measured invasively by direct cannulation of the portal vein with a micro-tip pressure monitor at the end of the study. Liver fibrosis was evaluated by histology, biochemical determination of collagen and analysis of profibrogenic gene expression by qRT-PCR.

Results of PSC Model:

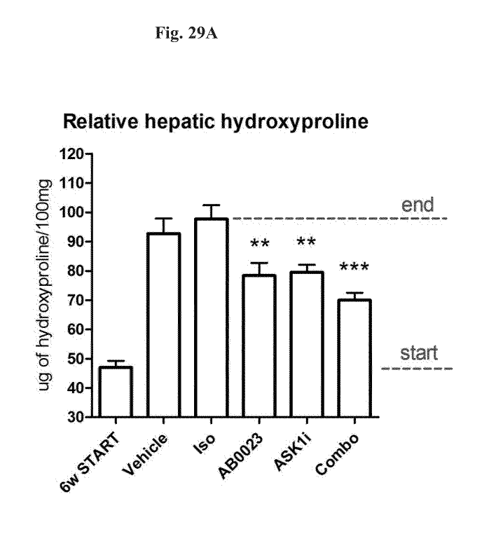

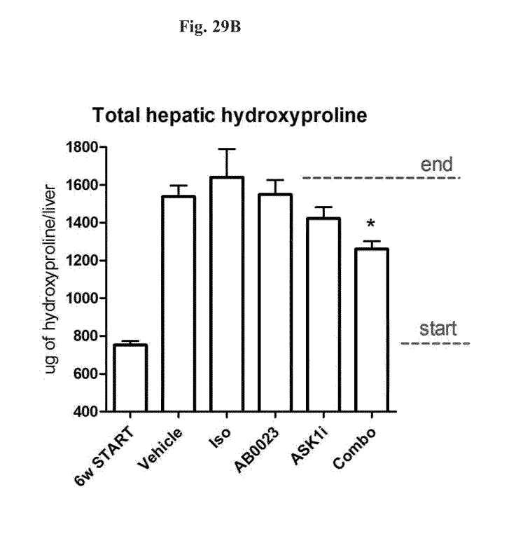

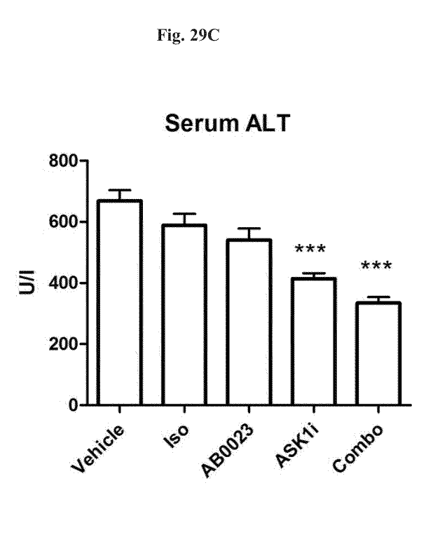

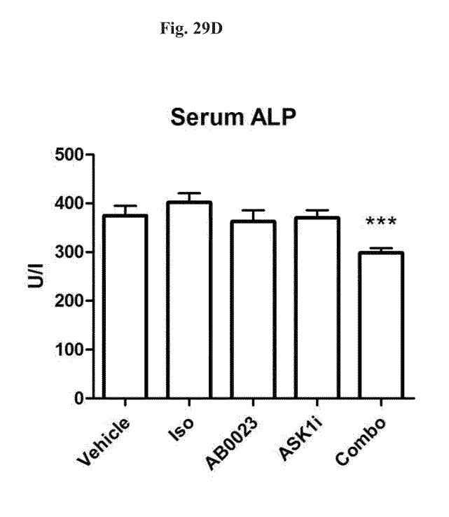

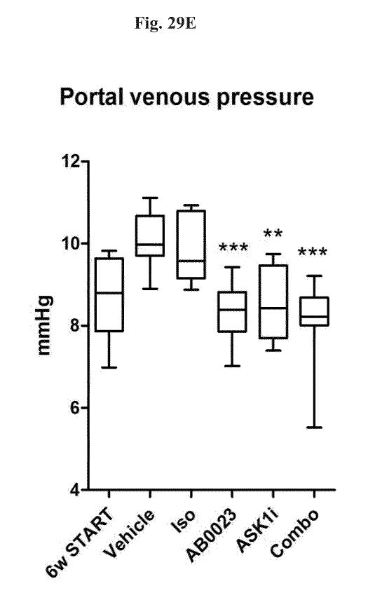

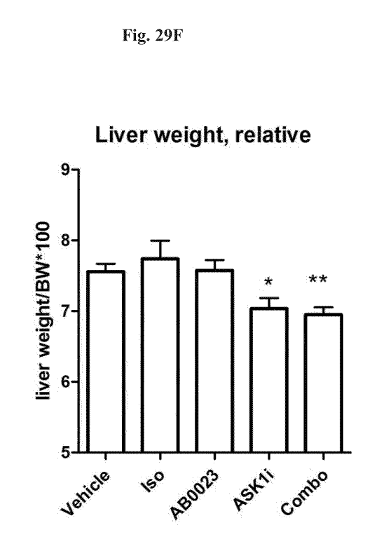

Compared to those of the control group, the mice treated with either Compound 1 or AB0023 exhibited reduced hepatic collagen deposition by 37 and 38% of hydroxyproline levels, respectively (p<0.01) and the mice treated with both Compound 1 and AB0023 exhibited reduced hepatic collagen deposition by 55% (p<0.001) (FIGS. 29A and 29B). In addition, the mice treated with Compound 1 or the combination of Compound 1 and AB0023 exhibited reduced serum ALT levels by 38% and 50%, respectively, compared to those of the control group (FIG. 29C). Only the mice received the combination treatment exhibited reduction in serum ALP levels (FIG. 29D).

At the end of the study, the control group developed portal hypertension (10.1±0.2 compared to 8.8±0.5 mmHg at 6 weeks of age prior to treatment). On the other hand, all treatment groups exhibited no increases in portal pressure since the start of treatment. The mice received the combination treatment exhibited lowest average PVP of 8.11±0.3 mmHg, and the mice received AB0023 or Compound 1 exhibited PVP of 8.36±0.23 mmHg and 8.52±0.3 mmHg, respectively (FIG. 29E). Moreover, the groups received Compound 1 or the combination treatment showed decreased liver weight (FIG. 29E). The results of the PSC model indicated that ASK1 inhibitor or LOXL2 inhibitor as a single agent inhibit or prevent fibrosis progression and portal hypertension, and that the combination of ASK1 inhibitor and LOXL2 inhibitor increases the inhibition or prevention of biliary fibrosis progression while continuing to inhibit advancement of portal hypertension.

Results of the NASH Model:

The group treated with Compound 1 resulted in a 33% reduction of hepatic steatosis, a 44% reduction in hepatic HYP content, and an 84% reduction in percent collagen area by 2^{nd} harmonic imaging.

The effects of Compound 1 were further increased in combination with AB0023. The level of tissue collagen was reduced relative to those at the start of treatment controls, suggesting reversal of established disease. Compound 1 treatment led to statistically significant improvements in AST/ALT, serum cholesterol, and cholesterol/triglyceride. Both treatments of Compound 1 alone and AB0023 alone affected synthesis rates of several extracellular matrix proteins (Col1a1, Col1a3, and Col1a5) involved in scarring.

The group treated with Compound 1 alone exhibited body weight loss without affecting food consumption or caloric intake. This indicates that Compound 1 treatment reversed insulin resistance, by normalizing fasting blood glucose and insulin levels by 17.1% and 13.7% respectively. Similar results were observed in the group treated with AB0023 alone. In additional studies, the group treated AB0023 did not affect in blood glucose and insulin levels. The group treated with Compound 1+AB0023 exhibited lower hepatic HYP levels compared to the groups treated with Compound 1 alone or AB0023 alone.

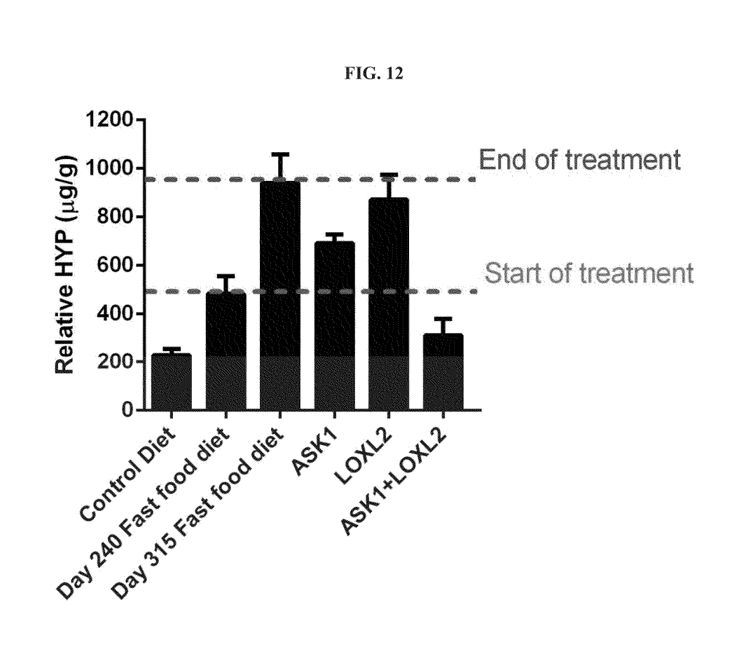

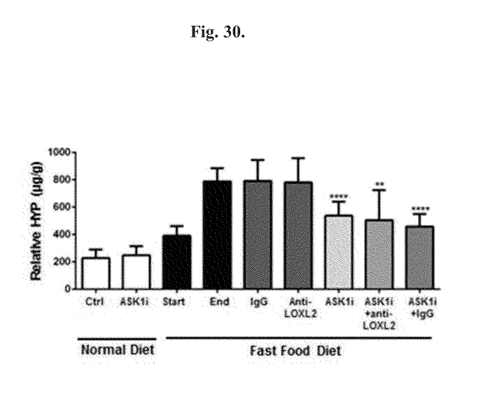

As shown in FIG. 12, when ASK1 inhibitor Compound 1 and LOXL2 inhibitor AB0023 (the murine anti-LOXL2 antibodies, see U.S. Pat. No. 8,461,303) were administered after 240 days of a high fat diet, the decrease in relative hydroxyproline levels (as a measure of liver collagen content) was more than additive when compared to the difference in hydroxyproline levels after administration of an ASK1 inhibitor or a LOXL2 inhibitor alone. Additional studies showed that the treatments with Compound 1 alone resulted in decrease in relative hydroxyproline levels, whereas the treatment of AB0023 alone did not result in a decrease in relative hydroxyproline levels and the combination of Compound 1 and AB0023 did not result in a further decrease in relative hydroxyproline levels (FIG. 30).

The results show that with the treatment with ASK1 and LOXL2 inhibitors reversed fibrosis in two preclinical models of human liver disease. Also, the treatment with ASK1 inhibitor alone led to improvements in metabolic parameters associated with human NASH. These data support the therapeutic use of an ASK1 inhibitor in combination with LOXL2 inhibitors.

### Example 4

### Effects of ASK1 Inhibitor in the Interventional NASH or PSC Models

Interventional NASH Model:

Commercially available high fat, high cholesterol, high sugar diet was administered continuously to 12 week old male C57BL/6 mice for 320 days. At day 240, Compound 1 was administered in chow 0.15% by weight). At 80 days of treatment, collagen synthesis and HYP levels in each group (n=15) were determined using D₂O labeling. Aliquots of liver (200 mg) were incubated for 16 hrs in 3 mL of 6 M HCl at 116° C. Liver homogenate (1.5 mL) was centrifuged at 14,000×rpm for 10 min and the resulting supernate was d diluted 1:1 with 4 M HCl. HYP levels were quantified using a commercially available hydroxyproline assay (Quickzyme Biosciences).

Interventional PSC Model:

12-week old Mdr2^{-/-} were treated with Compound 1. After 56 days of treatment, liver histology and tissue collagen content of each group (n=20) were determined.

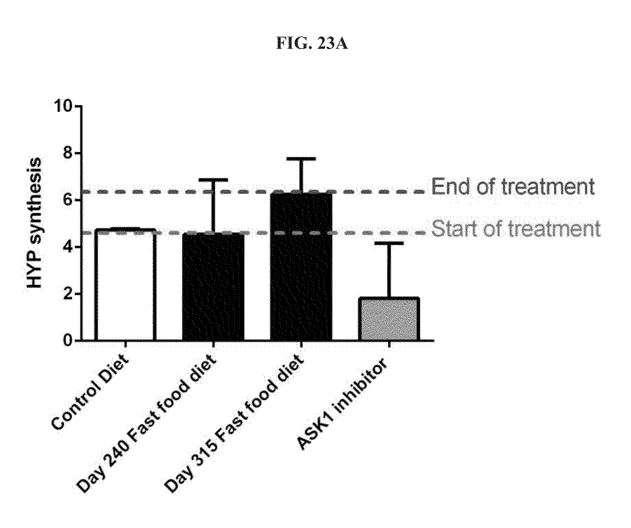

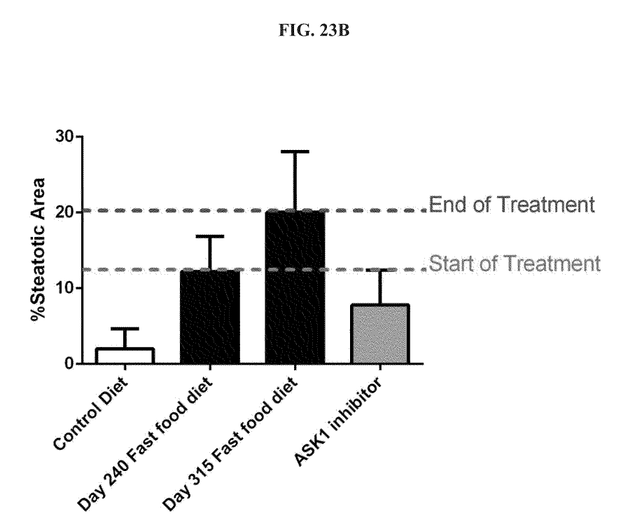

As shown in FIG. 12 and FIG. 23A, the mice treated with Compound 1 exhibited reduced hepatic hydroxyproline levels from 765.3 μg/g at day 0 of the treatment to 612 μg/g at day 80 of the treatment, a 44% reduction. Also, in the mice treated with Compound 1, hydroxyproline synthesis was reduced from 71% to 6.3% at day 80 of the control group, to 1.8% at day 80 of the group treated with compound 1. ASK1 inhibition decreased hepatic steatosis from 20.1% to 7.8% positive steatotic area, a 61.9% reduction (FIG. 23B). Statistically less steatosis was observed after the Compound 1 treatment when compared to day 0 of the control group.

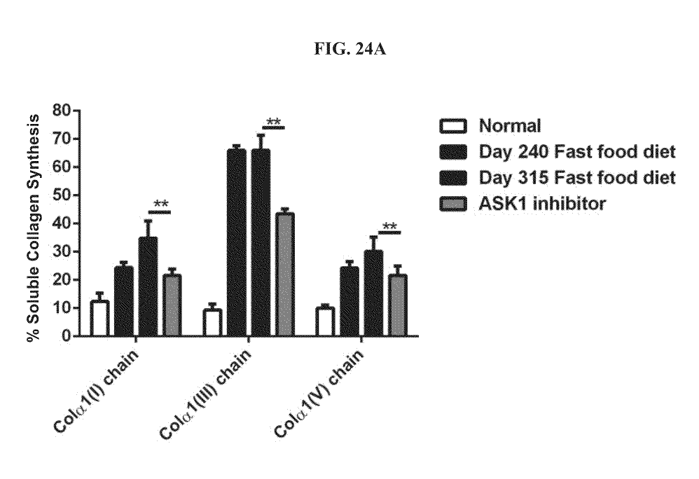

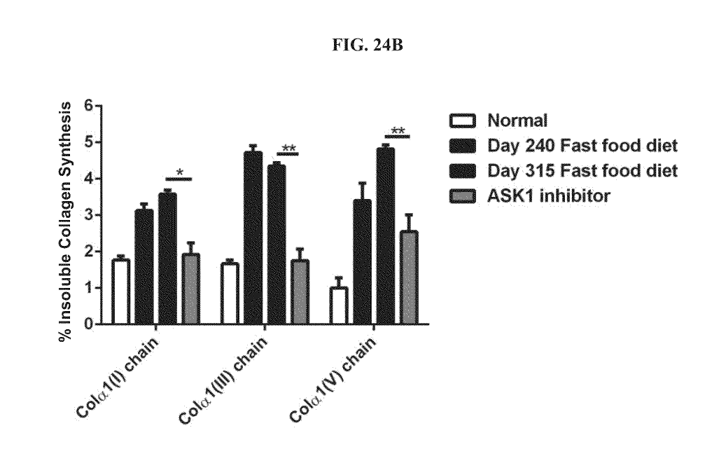

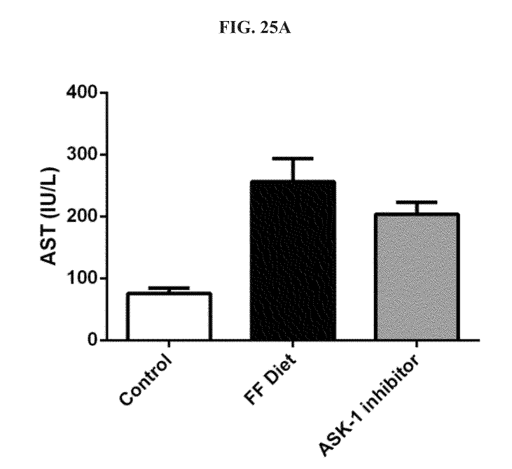

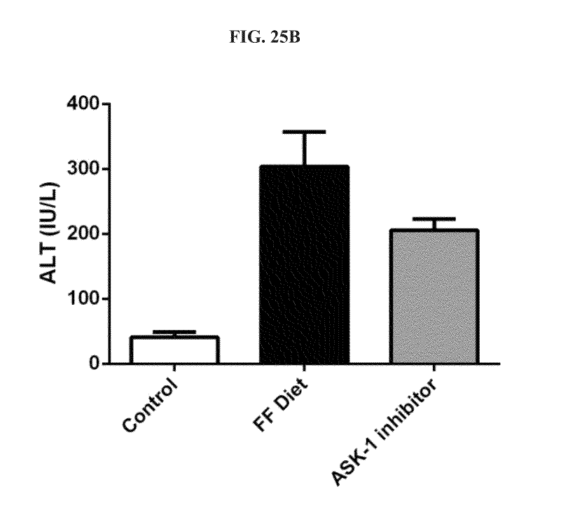

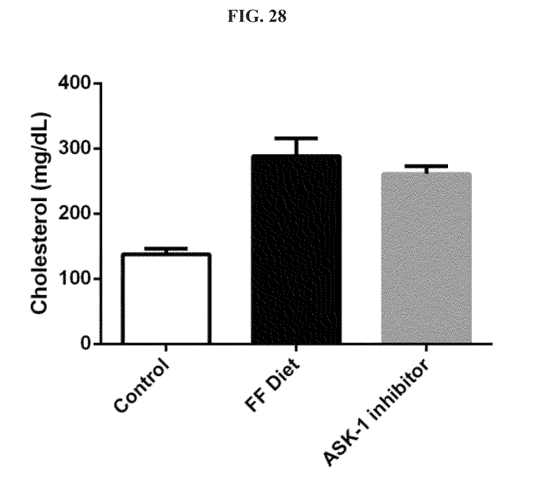

In addition, the treatment with Compound 1 reduced the circulating levels of AST by 20.5% (FIG. 25A), ALT by 32.3% (FIG. 25B), and cholesterol by 9.4% (FIG. 28) compared to those of the controls (i.e. no Compound 1 treatment). As shown in FIG. 24A, the treatment of Compound 1 reduced synthesis of several extracellular matrix proteins, Colα1(I), Colα1(III), Colα1(V), by 38.2%, 33.9%, and 28.2%, respectively, in the soluble fractionand and by 46.2%, 59.8%, and a 47.1%, respectively, in the insoluble fraction (FIG. 24B). A decreased expression of p-p38 in the Compound 1-treated animals was also observed (data not shown).

Additional analyses were summarized in Table 2. The results showed that Compound 1 reduced synthesis rates of COL1α(I), COL1α(III), and COLα1(V) by 31%, 37%, and 45%, respectively, in the soluble fraction and by 38%, 38% and 60%, respectively, in the insoluble fraction (Table 2).

TABLE 2
Synthesis of HYP, αSMA, COL1α(I), COL1α(III),
and COLα1(V) in the groups receiving normal diet,
NASH diet for 240 or 315 days, or NASH diet with Compound 1.

| | | NASH Diet^{b} | NASH Diet^{b} | 0.15% |
|---|---|---|---|---|
| | Normal Diet | (Day 240) | (Day 315) | Compound 1^{c} |
| HYP | 132 ± 35.9^{a} | 483 ± 72.1 | 765.3 ± 10 | 612 ± 109 |
| HYP synthesis | 4.7 ± 0.1% | 7.5 ± 3.9% | 9.9 ± 3.7% | 5.0 ± 1.9% |
| αSMA synthesis | 32.3 ± 3.1% | 69.3 ± 5.2% | 77.1 ± 4.5% | 59.6 ± 6.3% |
| Soluble COL1α | 12.4 ± 2.9% | 22 ± 5.9% | 26 ± 6.1% | 18.7 ± 5.7% |
| (I) synthesis | | | | |
| Soluble COL1α | 9.3 ± 2.1% | 38.7 ± 1.8% | 40.6 ± 1.5% | 25.7 ± 1.2% |
| (III) synthesis | | | | |
| Soluble COLα1 | 4.4 ± 0.3% | 11.9 ± 2.6% | 19.5 ± 6.4% | 10.7 ± 3.1% |
| (V) synthesis | | | | |
| Insoluble COL1α | 1.8 ± 0.1% | 3.6 ± 0.2% | 4.9 ± 0.3% | 3 ± 0.7% |
| (I) synthesis | | | | |
| Insoluble COL1α | 3.7 ± 0.6% | 6.2 ± 0.2% | 7.9 ± 3.4% | 4.9 ± 2.0% |
| (III) synthesis | | | | |
| Insoluble COLα1 | 1.8 ± 0.3% | 7.3 ± 0.4% | 11.9 ± 1.3% | 4.8 ± 0.4% |
| (V) synthesis | | | | |

^{a}all units in μg/ml
^{b}NASH diet: fast food diet
^{c}NASH diet with 0.15% Compound 1 at Day 315

Also, ELISA kits were used to determine the effects of Compound 1 on serum levels of metalloproteinase-1 (TIMP1), hyalronan (HA), osteopontin (OPN), and interleukin-6 (IL-6). The results are summarized in Table 3. In the mice treated with Compound 1, levels of TIMP1 and HA were reduced by 41% (i.e. 3105±884.6 vs. 1841±532.3 pg/ml) and by 26% (i.e. 922.2±141.3 vs. 680.9±181.3 ng/ml), respectively. Also, in the mice treated with Compound 1, levels of OPN and IL-6 were reduced by 33% (i.e. 92.5±42.3 vs. 61.9±23 ng/ml) and by 35% (i.e. 21±6.7 vs. 13.7±7 pg/ml), respectively (Table 3).

In addition, immunohistochemistry and immunoblot analysis were used to characterize the levels of phospho-p38 (p-p38), phospho-JNK1 (p-JNK1), and phospho-MKK4 (p-MKK4) proteins. The results of immunoblot analysis are summarized in Table 3. The results showed that the treatment with 0.15% of compound 1 reduced the activation of p38. Compared to untreated mice, the mice treated with Compound 1 had reduced levels of phospho-p38 (p-p38) (i.e. 0.2±0.1 vs. 1.1±0.04) (Table 3). Other downstream markers of ASK1 activation, including phosphorylated c-JUN kinase 1 (pJNK1) and phosphorylated mitogen activated kinase kinase 4 (p-MKK4) were also reduced in the mice treated with 0.15% of Compound 1. Levels of p-JNK1 and p-MKK4 in the treated mice were reduced by 86% (0.2±0.1 vs. 1.5±0.1) and by 53% (0.7±0.09 vs. 1.5±0.2) compared to the untreated mice (Table 3).

TABLE 3
Levels of TIMP1, HA, OPN, IL-6, p-p38, p-JNK1, p-MKK4 in the groups receiving
normal diet, NASH diet for 240 or 315 days, or NASH diet with Compound 1.

| | | NASH Diet | NASH Diet | 0.15% |
|---|---|---|---|---|
| | Normal Chow | (Day 240) | (Day 315)^{c} | Compound 1^{d} |
| TIMP1 | 1111 ± 97.5 | 2574 ± 170.8 | 3105 ± 884.6 | 1841 ± 532.3 |
| | pg/mL | pg/mL | pg/mL | pg/mL |
| HA | 524.9 ± 45.8 | 659.5 ± 112.1 | 922.2 ± 141.3 | 680.9 ± 181.3 |
| | ng/mL | ng/mL | ng/mL | ng/mL |
| OPN | 43.9 ± 3 | 89.8 ± 42.3 | 92.5 ± 42.3 | 61.9 ± 23 |
| | ng/mL | ng/mL | ng/mL | ng/mL |
| IL-6 | ND^{a} | 19 ± 8 | 21 ± 6.7 | 13.7 ± 7 |
| | | pg/mL | pg/mL | pg/mL |
| p-p38 | 1.1 ± 0.03^{b} | ND^{a} | 1.1 ± 0.04^{b} | 0.2 ± 0.1^{b} |
| p-JNK1 | 1.0 ± 0.04^{b} | ND^{a} | 1.5 ± 0.1^{b} | 0.2 ± 0.1^{b} |
| p-MKK4 | 1.2 ± 0.1^{b} | ND^{a} | 1.5 ± 0.2^{b} | 0.7 ± 0.09^{b} |

^{a}ND: below levels of quantification
^{b}Normalized intensity determined by the immunoblot analysis
^{c}NASH diet: fast food diet
^{d}NASH diet with 0.15% Compound 1 at Day 315

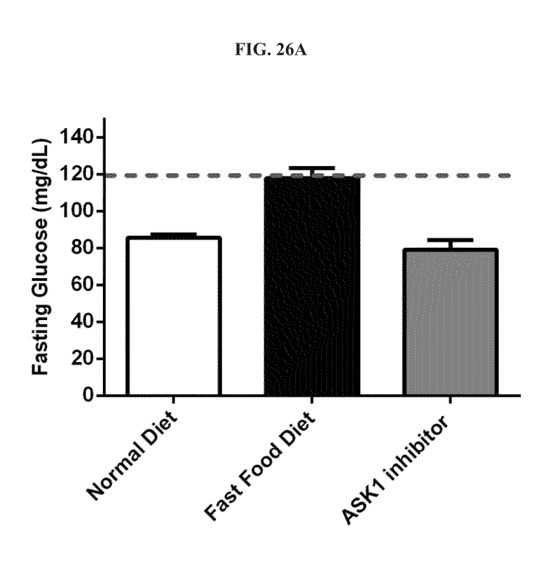

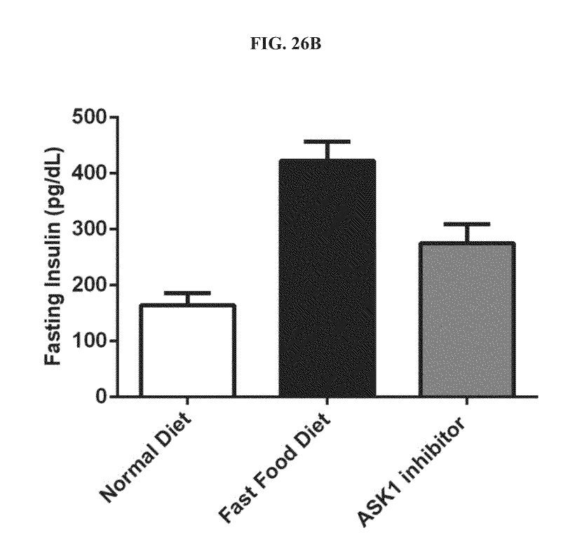

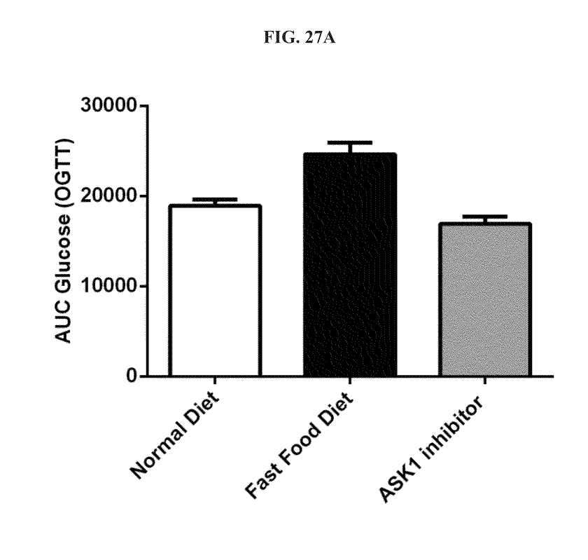

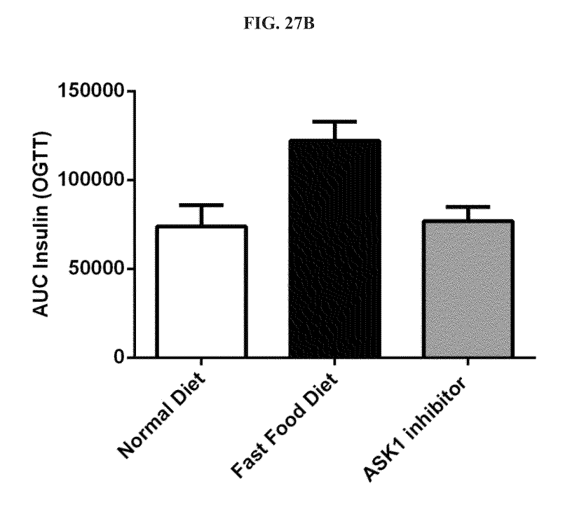

The mice maintained on fast food diet for 315 days developed fasting hyperglycemia and fasting hyperinsulinemia, whereas those treated with Compound 1 exhibited reduced fasting blood glucose levels from 117.8 mg/dL to 79.1 mg/dL (FIG. 26A) and reduced fasting insulin levels from 422.1 pg/dL to 274.6 pg/dL (FIG. 26B). In addition, fast food diet fed mice developed defective glucose metabolism as measured during an oral glucose tolerance test, whereas those treated with Compound 1 exhibited improved glucose metabolism and insulin resistance (FIG. 27A and FIG. 27B).

The results suggest that ASK1 inhibitor reversed pre-formed fibrosis and improved metabolic parameters associated with NASH. This suggest that ASK1 inhibitor (such as the compounds described herein) would provide potential metabolic effects, for example, modulating glucose parameters (i.e. fasting glucose, HbA1c, oral glucose tolerance test), reducing non-fasting glucose, improving insulin resistance, and/or reducing weight gain due to obesity.

### Example 5

### ASK1 Pathway in the Livers of NASH Patients

Liver biopsy samples were obtained from healthy human subjects (n=8) and NASH patients with cirrhosis (n=9) or fibrosis at different stages (n=9). These biopsies were staged for fibrosis using an established system described in Brunt et al., (Am J Gastroenterol 1999; 94 (9):2467-74). Briefly, stage 0 indicates no fibrosis (NAFLD only), stage 1 indicates enlargement of the portal areas by fibrosis, stage 2 indicates fibrosis extending out from the portal area with rare bridges between portal areas, stage 3 indicates many bridges between portal areas, and stage 4 indicates cirrhosis. In this study, five samples were graded as F1 fibrosis, four samples were graded as F3 fibrosis.

Quantitative real time polymerase chain reaction (qRT-PCR) was used to determine the expression levels of thioredoxin interacting protein (TxNIP), thioredoxin reductase 1(Trx1) and 2 (Trx2), TGF-β1, TGF-β2, TGF-β3, αSMA, COL1α1, COL3α1, and HDAC10 (control). TGF-β1 was previously shown to activate ASK1 and fibrogenesis, whereas Trx1 and Trx2 were shown to inhibit ASK1 and TxNIP was shown to activate ASK1.

The results are summarized in Table 4 and suggests a correlation of TxNIP levels to TGF-β levels, which is consistent with previous studies (Perrone et al., Cell death & disease 2010; 1:e65). Additionally, the results showed that all NASH patients, regardless of disease severity, had increased levels of TxNIP and reduced levels of Trx1.

TABLE 4
Levels of TxNIP, Trx1, TGF-β1, αSMA, COL1α1 in healthy
subjects or NASH patients having fibrosis or cirrhosis.

| Samples | TxNIP | Trx1 | TGF-β1 | αSMA | COL1a1 |
|---|---|---|---|---|---|
| Healthy | 0.89 ± 0.18 | 1.4 ± 0.4 | 10.2 ± 4.1 | 4.5 ± 2.4 | 4.8 ± 3.4 |
| NASH | 6.5 ± 2.3 | 0.26 ± 0.14 | 10.2 ± 4.1 | 4.5 ± 2.4 | 4.8 ± 3.4 |
| Fibrosis | | | | | |
| NASH | 3.9 ± 3.4 | 0.22 ± 0.12 | 6.7 ± 11.5 | 6.9 ± 3.6 | 2.8 ± 2.7 |
| Cirrhosis | | | | | |

In addition, p-p38 levels in the liver samples of NASH patients (n=13) were determined by immunohistochemistry. The results showed p-p38 staining in hepatocytes and/or inflammatory cells including Kupffer cells in 12 of 13 patient liver samples (data not shown). This indicates that the ASK1 pathway was activated in the livers of NASH patients.

### Example 6

### Treating NASH Patients with ASK1 and/or LOXL2 Inhibitors

Chronic liver disease and the subsequent end stage liver disease are increasing despite improved prevention and treatment of viral hepatitis. This may be due to the emerging epidemic of obesity and metabolic syndrome, which may result an increased incidence of NASH. Over time, NASH results in progressive liver fibrosis, resulting in cirrhosis of the liver. Approximately 50% of patients with NASH have advanced liver fibrosis (bridging fibrosis or cirrhosis), which is associated with increased morbidity and mortality (Yeh et al., Gastroenterology, 147 (4):754-764 (2014)). Cirrhosis would increase the risk of patients develop hepatocellular carcinoma (HCC) and other complications of end stage liver disease, including jaundice, fluid retention (edema and ascites), portal hypertension and variceal bleeding, impaired coagulation and hepatic encephalopathy. Decompensated liver disease, which is commonly defined by the development of one of the above complications, would lead to a high mortality and the only known effective treatment is liver transplantation. With the increasing prevalence of obesity and obesity-related diseases, NASH may become the leading indication for liver transplantation, and the leading etiology of HCC among liver transplant recipients in the US as well as worldwide (Wree et al., Nature Reviews Gastroenterology & hepatology, 10 (11):627-636 (2013); Afzali et al., Liver Transpl., 18 (1):29-37 (2012)). In the United States, an estimated 16 million adults have NASH (Vernon et al., Aliment Pharmacol. Ther., 34 (3):274-285 (2011)). As approved therapies are not currently available, there is an unmet medical need for NASH treatment.

Without being bound by any hypothesis, the primary cause of NASH may be the metabolic syndrome which may be characterized by the impact of obesity, insulin resistance, and/or hypercholesterolemia in the liver. Fatty liver or simple steatosis may not be sufficient to cause liver injury. In addition to steatosis, inflammation and fibrosis may cause NASH and result in the progression to end stage liver disease and/or other complications. NASH may be resulted from the setting of steatosis and metabolic dysfunction, increased oxidative stress and the generation of reactive oxygen species (ROS), which may mediate the inflammatory changes in the liver (steatohepatitis) with progressive liver fibrosis (Koek et al., Clin. Chim. Acta, 412:1297-305 (2011); Sumida et al., Free Radical Research, 47 (11):869-880 (2013)). The potential pathways associated with the disease progression of NASH may include those involved in metabolic dysfunction in the hepatocyte, activation of hepatic stellate cells and macrophages leading to progressive inflammation and liver fibrosis. Advanced fibrosis and cirrhosis may be characterized by extensive collagen deposition and remodeling of the extracellular matrix.

AB0024 is a humanized monoclonal antibody with an immunoglobulin gamma 4 (IgG4) isotype directed against human lysyl oxidase like molecule 2 (LOXL2). LOXL2 is a secreted copper-dependent amine oxidase and catalyzes the first step in the cross linking of collagen and elastin, leading to remodeling of the extracellular matrix (Payne et al., J. Cell Biochem., 101 (6):1338-1354 (2007)). Without being bound to any hypothesis, a LOXL2 inhibitor, such as anti-LOXL2 antibodies, may inhibit the cross-linking of hepatic collagen, disrupting the process of fibrogenesis within the liver and shifting the liver to a fibrosis regression state, leading to a reduction in intrahepatic collagen, deactivation of hepatic stellate cells, a decrease in pathologic fibrosis stage, and/or reversal of fibrosis, resulting an improved clinical outcome.

As shown above, ASK1 inhibitor, such as Compound 3, and LOXL2 inhibitor, such as AB0024, would reduce fibrosis in the animal models of advanced liver fibrosis. Further studies are conducted to investigate the clinical efficacy. Suitable subjects will receive placebo, Compound 3 (6 mg or 18 mg, once daily, orally), AB0024 (125 mg, once a week, subcutaneously), or Compound 3 (6 mg or 18 mg, once daily, orally) in combination with AB0024 (125 mg, once a week, subcutaneously) for a period of 24 weeks. Suitable subjects are those having NASH and advanced fibrosis but not cirrhosis, as diagnozed by liver biopsy, MRI-PDFF (magnetic resonance imaging—proton density fat fraction), MRE (magnetic resonance elastography) or Fibroscan.

The subjects will be monitored at various treatment points; for example, 4, 8, 12, 16, 20, and/or 24 weeks after treatment. The studies monitor several variables, including MRI-PDFF, MRE or Fibroscan, the non-invasive measures of fibrosis and steatosis (such as change from baseline in MRI-PDFF to assess reduction in steatosis, change from baseline in liver stiffness by MRE or Fibroscan to assess reduction in liver fibrosis), histology (such as change from baseline in the NAS and Brunt/Kleiner fibrosis scores, reduction in the amount of activated hepatic stellate cells as assessed by change from baseline in α-smooth muscle actin on liver biopsy), change from baseline in non-invasive markers of fibrosis including the ELF™ test score and FibroSURE/FibroTest, reduction in fibrosis disease activity as assessed by change from baseline in serum LOXL2 levels, change from baseline in markers of liver injury and function: ALT, AST, bilirubin, GGT and alkaline phosphatase, change from baseline in HOMA-IR, serum lipid profiles, and HbA1c levels, change from baseline in collagen turnover and lipid biogenesis (heavy water labeling), change in body weight from baseline, and/or inhibition of the ASK-1 pathway in the liver by RNA expression profiling and immunohistochemistry (p-p38, p-ASK).

All of the U.S. patents, U.S. patent application publications, U.S. patent applications, foreign patents, foreign patent applications and non-patent publications referred to in this specification are incorporated herein by reference, in their entirety to the extent not inconsistent with the present description. From the foregoing it will be appreciated that, although specific embodiments of the invention have been described herein for purposes of illustration, various modifications may be made without deviating from the spirit and scope of the present application.

Below is a listing of sequences described throughout the specification.

| SEQ ID | |
|---|---|
| NO: | Sequence |
| 1 | VRLRGGAYIGEGRVEVLKNGEWGTVCDDKWDLVSASVVCRELGFGSAK |
| | EAVTGSRLGQGIGPIHLNEIQCTGNEKSIIDCKFNAESQGCNHEEDAGVRC |
| | NTPAMGLQKKLRLNGGRNPYEGRVEVLVERNGSLVWGMVCGQNWGIVE |
| | AMVVCRQLGLGFASNAFQETWYWHGDVNSNKVVMSGVKCSGTELSLAH |
| | CRHDGEDVACPQGGVQYGAGVACS |
| 2 | QVQLVQSGAELKKPGASVKVSCKASGYAFTYYLIEWVKQAPGQGLE |
| | WIGVINPGSGGTNYNEKFKGRATLTADKSTSTAYMELSSLRSEDSA |
| | VYFCARNWMNFDYWGQGTTVTVSS |
| 3 | QVQLVQSGAEVKKPGASVKVSCKASGYAFTYYLIEWVRQAPGQGLE |
| | WIGVINPGSGGTNYNEKFKGRATLTADKSTSTAYMELSSLRSEDTA |
| | VYFCARNWMNFDYWGQGTTVTVSS |
| 4 | QVQLVQSGAEVKKPGASVKVSCKASGYAFTYYLIEWVRQAPGQGLE |
| | WIGVINPGSGGTNYNEKFKGRATITADKSTSTAYMELSSLRSEDTA |
| | VYFCARNWMNFDYWGQGTTVTVSS |
| 5 | QVQLVQSGAEVKKPGASVKVSCKASGYAFTYYLIEWVRQA |
| | PGQGLEWIGVINPGSGGTNYNEKFKGRVTITADKSTSTAYMELSSLR |
| | SEDTAVYYCARNWMNFDYWGQGTTVTVSS |
| 6 | DIVMTQTPLSLSVTPGQPASISCRSSKSLLHSNGNTYLYWFLQKPGQSPQFL |
| | IYRMSNLASGVPDRFSGSGSGTAFTLKISRVEAEDVGVYYC |
| | MQHLEYPYTFGGGTKVEIK |
| 7 | DIVMTQTPLSLSVTPGQPASISCRSSKSLLHSNGNTYLYWFLQKPG |
| | QSPQFLIYRMSNLASGVPDRFSGSGSGTDFTLKISRVEAEDVGVYYC |
| | MQHLEYPYTFGGGTKVEIK |
| 8 | DIVMTQTPLSLSVTPGQPASISCRSSKSLLHSNGNTYLYWYLQKPG |
| | QSPQFLIYRMSNLASGVPDRFSGSGSGTDFTLKISRVEAEDVGVYYC |
| | MQHLEYPYTFGGGTKVEIK |
| 9 | GYAFTYYLIE |
| 10 | VINPGSGGTNYNEKFKG |
| 11 | NWMNFDY |
| 12 | RSSKSLLHSNGNTYLY |
| 13 | RMSNLAS |
| 14 | MQHLEYPYT |
| 15 | MEWSRVFIFLLSVTAGVHSQVQLQQSGAELVRPGTSVKVSCKASGYAFTY |
| | YLIEWVKQRPGQGLEWIGVINPGSGGTNYNEKFKGKATLTADKSSSTAY |
| | MQLSSLTSDDSAVYFCARNWMNFDYWGQGTTLTVSS |
| 16 | MRCLAEFLGLLVLWIPGAIGDIVMTQAAPSVSVTPGESVSISCRSSKSLLHS |
| | NGNTYLYWFLQRPGQSPQFLIYRMSNLASGVPDRFSGSGSGTAFTLRISRV |
| | EAEDVGVYYCMQHLEYPYTFGGGTKLEIK |

*CDRs are bolded.

What is claimed is:

1. A method of treating and/or preventing liver disease in a patient in need thereof, comprising administering to the patient a therapeutically effective amount of an ASK1 inhibitor.

2. The method of claim 1, wherein the liver disease is selected from the group consisting of chronic liver disease, metabolic liver disease, steatosis, liver fibrosis, primary sclerosing cholangitis (PSC), cirrhosis, liver fibrosis, non-alcoholic fatty liver disease (NAFLD), non-alcoholic steatohepatitis (NASH), hepatic ischemia reperfusion injury, primary biliary cirrhosis (PBC), and hepatitis.

3. The method of claim 1, wherein the ASK1 inhibitor is a compound of formula (I): 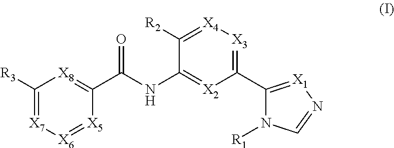
wherein:
R¹ is alkyl, alkenyl, alkynyl, cycloalkyl, aryl, heteroaryl, or heterocyclyl, wherein the alkyl, alkenyl, alkynyl, cycloalkyl, aryl, heteroaryl, and heterocyclyl are optionally substituted with from one to three substituents selected from halo, oxo, alkyl, cycloalkyl, heterocyclyl, aryl, aryloxy, —NO₂, R⁶, —C(O)—R⁶, —OC(O)—R⁶—C(O)—O—R⁶, C(O)—N(R⁶)(R⁷), —OC(O)—N(R⁶)(R⁷), —S—R⁶, —S(═O)R⁶, —S(═O)₂R⁶, —S(═O)₂—N(R⁶)(R⁷), —S(═O)₂—O—R⁶, —N(R⁶)(R⁷), —N(R⁶)—C(O)—R⁷, —N(R⁶)—C(O)—O—R⁷, —N(R⁶)—C(O)—N(R⁶)(R⁷), —N(R⁶)—S(═O)₂—R⁶, —CN, and —O—R⁶, and wherein the alkyl, cycloalkyl, heterocyclyl, phenyl, and phenoxy are optionally substituted by from one to three substituents selected from alkyl, cycloalkyl, alkoxy, hydroxyl, and halo; wherein R⁶ and R⁷ are independently selected from the group consisting of hydrogen, (C1-C15) alkyl, cycloalkyl, heterocyclyl, aryl, and heteroaryl, all of which are optionally substituted with from one to three substituents selected from halo, alkyl, monoalkylamino, dialkylamino, alkyl amide, aryl amide, heteroaryl amide, —CN, lower alkoxy, —CF₃, aryl, and heteroaryl; or
R⁶ and R⁷ when taken together with the nitrogen to which they are attached form a heterocycle;
R² is hydrogen, halo, cyano, alkoxy, or alkyl optionally substituted by halo;
R³ is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, —NO₂, haloalkyl, haloalkoxy, —CN, —O—R⁶, —O—C(O)—R⁶, —O—C(O)—N(R⁶)(R⁷), —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—R⁶, —C(O)—N(R⁶)(R⁷), and —N(R⁶)—S(═O)₂—R⁷, wherein the alkyl, alkoxy, cycloalkyl, aryl, heteroaryl or heterocyclyl is optionally substituted with from one to five substituents selected from halo, oxo, —NO₂, alkyl, haloalkyl, haloalkoxy, —N(R⁶)(R⁷), —C(O)—R⁶, —C(O)—O—R⁶, —C(O)—N(R⁶)(R⁷), —CN, —O—R⁶, cycloalkyl, aryl, heteroaryl and heterocyclyl; with the proviso that the heteroaryl or heterocyclyl moiety includes at least one ring nitrogen atom;
X¹, X², X³, X⁴, X⁵, X⁶, X⁷ and X⁸ are independently C(R⁴) or N, in which each R⁴ is independently hydrogen, alkyl, alkoxy, cycloalkyl, aryl, heteroaryl, heterocyclyl, halo, —NO₂, haloalkyl, haloalkoxy, —CN, —O—R⁶, —S—R⁶, —N(R⁶)(R⁷), —S(═O)—R⁶, —S(═O)₂R⁶, —S(═O)₂—N(R⁶)(R⁷), —S(═O)₂—O—R⁶, —N(R⁶)—C(O)—R⁷, —N(R⁶)—C(O)—O—R⁷, —N(R⁶)—C(O)—N(R⁶)(R⁷), —C(O)—R⁶, —C(O)—O—R⁶, —C(O)—N(R⁶)(R⁷), or —N(R⁶)—S(═O)₂—R⁷, wherein the alkyl, cycloalkyl, aryl, heteroaryl, and heterocyclyl is further optionally substituted with from one to five substituents selected from halo, oxo, —NO₂, —CF₃, —O—CF₃, —N(R⁶)(R⁷), —C(O)—R⁶, —C(O)—O—R⁷, —C(O)—N(R⁶)(R⁷), —CN, —O—R⁶; or
X⁵ and X⁶ or X⁶ and X⁷ are joined to provide optionally substituted fused aryl or optionally substituted fused heteroaryl; and
with the proviso that at least one of X², X³, and X⁴ is C(R⁴); at least two of X⁵, X⁶, X⁷, and X⁸ are C(R⁴); and at least one of X², X³, X⁴, X⁵, X⁶, X⁷ and X⁸ is N;
or a pharmaceutically acceptable salt, isomer, stereoisomer, or tautomer thereof.

4. The method of claim 1, wherein the ASK1 inhibitor is a compound of formula (II): 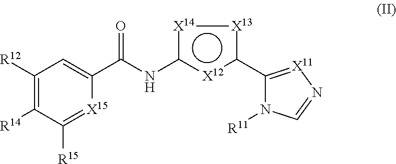
wherein:
R¹¹ is (C1-C6)alkyl, (C1-C6)alkenyl, (C1-C6)alkynyl, (C3-C6)cycloalkyl, aryl, heteroaryl, or heterocyclyl, wherein the (C1-C6)alkyl, (C1-C6)alkenyl, (C1-C6)alkynyl, (C3-C6)cycloalkyl, aryl, heteroaryl, and heterocyclyl are optionally substituted with from one to four substituents selected from the group consisting of halo, hydroxyl, oxo, alkyl, cycloalkyl, heterocyclyl, aryl, aryloxy, NO₂, R¹⁶, C(O)R¹⁶, OC(O)R¹⁶C(O)OR¹⁶, C(O)N(R¹⁶)(R¹⁷), OC(O)N(R¹⁶)(R¹⁷), SR¹⁶, S(═O)R¹⁶, S(═O)₂R¹⁶, S(═O)₂N(R¹⁶)(R¹⁷), S(═O)₂OR¹⁶, N(R¹⁶)(R¹⁷), N(R¹⁶)C(O)R¹⁷, N(R⁶)C(O)OR¹⁷, N(R¹⁶)C(O)N(R¹⁶)(R¹⁷), N(R¹⁶)S(═O)₂R¹⁶, CN, and OR¹⁶, wherein the alkyl, cycloalkyl, heterocyclyl, aryl, and aryloxy are optionally substituted with from one to three substituents selected from alkyl, cycloalkyl, alkoxy, hydroxyl, and halo;
R¹⁶ and R¹⁷ are independently selected from the group consisting of hydrogen, (C1-C15)alkyl, cycloalkyl, heterocyclyl, aryl, and heteroaryl, wherein the (C1-C15)alkyl, cycloalkyl, heterocyclyl, aryl, and heteroaryl are optionally substituted with from one to three substituents selected from halo, alkyl, monoalkylamino, dialkylamino, alkyl amide, aryl amide, heteroaryl amide, CN, lower alkoxy, CF₃, aryl, and heteroaryl; or
R¹⁶ and R¹⁷ when taken together with the nitrogen to which they are attached form a heterocycle;
R¹² is aryl, heteroaryl, or heterocyclyl, wherein the aryl, heteroaryl, and heterocyclyl are optionally substituted with from one to five substituents selected from alkyl, alkoxy, cycloalkyl, cycloalkylalkyl, aryl, arylalkyl, heteroaryl, heteroarylalkyl, heterocyclyl, heterocyclylalkyl, halo, oxo, NO₂, haloalkyl, haloalkoxy, CN, OR¹⁶, OC(O)R¹⁶, OC(O)N(R¹⁶)(R¹⁷), SR¹⁶, N(R¹⁶)(R¹⁷), S(═O)R¹⁶, S(═O)₂R¹⁶, S(═O)₂N(R¹⁶)(R¹⁷), S(═O)₂OR¹⁶, N(R¹⁶)C(O)R¹⁷, N(R¹⁶)C(O)OR¹⁷, N(R¹⁶)C(O)N(R¹⁶)(R¹⁷), C(O)R¹⁶, C(O)OR¹⁶, C(O)N(R¹⁶)(R¹⁷), and N(R¹⁶)S(═O)₂R¹⁷, and wherein the alkyl, alkoxy, cycloalkyl, aryl, heteroaryl and heterocyclyl optionally substituted with one or more substituents selected from halo, oxo, NO₂, alkyl, haloalkyl, haloalkoxy, N(R¹⁶)(R¹⁷), C(O)R¹⁶, C(O)OR¹⁶, C(O)N(R¹⁶)(R¹⁷), CN, OR¹⁶, cycloalkyl, aryl, heteroaryl and heterocyclyl; with the proviso that the heteroaryl or heterocyclyl moiety includes at least one ring nitrogen atom;
R¹⁴ and R¹⁵ are independently hydrogen, halo, cyano, (C1-C6)alkyl, (C1-C6)alkoxy, or (C1-C6)cycloalkyl, wherein the alkyl, alkoxy, and cycloalkyl are optionally substituted by halo or (C3-C8)cycloalkyl;
X¹¹ and X¹⁵ are independently C(R¹³) or N, wherein each R¹³ is independently hydrogen, halo, (C1-C6)alkyl, (C1-C6)alkoxy or (C3-C8)cycloalkyl, wherein the alkyl and cycloalkyl are optionally substituted with from one to five substituents selected from halo, oxo, CF₃, OCF₃, N(R¹⁶)(R¹⁷), C(O)R¹⁶, C(O)OR¹⁷, C(O)N(R¹⁶)(R¹⁷), CN, and OR¹⁶; and
X¹², X¹³ and X¹⁴ are independently C(R¹³), N, O, or S; with the proviso that at least one of X¹², X¹³, and X¹⁴ is C(R¹³); and only one of X¹², X¹³, and X¹⁴ is O or S;
or a pharmaceutically acceptable salt, isomer, stereoisomer, or tautomer thereof.

5. The method of claim 1, wherein the ASK1 inhibitor is a compound selected from the group consisting of: 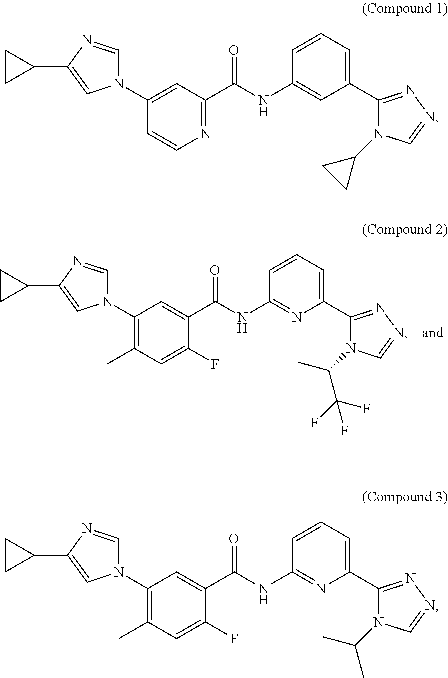 [SMILES: O=C(Nc1cccc(-c2nncn2C2CC2)c1)c1cc(-n2cnc(C3CC3)c2)ccn1 ; Cc1cc(F)c(C(=O)Nc2cccc(-c3nncn3[C@@H](C)C(F)(F)F)n2)cc1-n1cnc(C2CC2)c1 ; Cc1cc(F)c(C(=O)Nc2cccc(-c3nncn3C(C)C)n2)cc1-n1cnc(C2CC2)c1]
or a pharmaceutically acceptable salt thereof.

6. The method of claim 1, wherein the ASK1 inhibitor is administered orally.

7. The method of claim 1, wherein the ASK1 inhibitor is administered at 1, 2, 6, 10, 18, 20, 30, or 100 mg.

8. The method of claim 1, wherein the ASK1 inhibitor is administered once daily.

9. The method of claim 1, further comprising a therapeutically effective amount of a LOXL2 inhibitor.

10. The method of claim 9, wherein the LOXL2 inhibitor is an anti-LOXL2 antibody.

11. The method of claim 10, wherein the anti-LOXL2 antibody is a monoclonal anti-LOXL2 antibody or antigen-binding fragment thereof.

12. The method of claim 10, wherein the anti-LOXL2 antibody is a polyclonal anti-LOXL2 antibody or antigen-binding fragment thereof.

13. The method of claim 10, wherein the anti-LOXL2 antibody is an isolated antibody or antigen binding fragment thereof, comprising complementarity determining regions (CDRs), CDR1, CDR2, and CDR3, of a heavy chain variable region comprising the amino acid sequence set forth as SEQ ID NO: 2, 3, 4, or 5, and the CDRs, CDR1, CDR2, and CDR3, of a light chain variable region comprising the amino acid sequence set forth as SEQ ID NO: 6, 7, or 8, wherein the isolated antibody or antigen binding fragment thereof specifically binds a lysyl oxidase-like 2(LOXL2) protein.

14. The method of claim 13, wherein CDR1, CDR2, and CDR3 of the heavy chain variable region comprise the amino acid sequences set forth as SEQ ID NOs: 9, 10, and 11 respectively, and the CDR1, CDR2, and CDR3 of the light chain variable region comprise the amino acid sequences set forth as SEQ ID NOs: 12, 13, and 14, respectively.

15. The method of claim 10, wherein the anti-LOXL2 antibody has a heavy chain variable region comprising the amino acid sequence set forth as SEQ ID NO: 2, 3, 4, or 5, and a light chain variable region comprising the amino acid sequence set forth as SEQ ID NO: 6, 7, or 8, wherein the isolated antibody or antigen binding fragment thereof specifically binds a lysyl oxidase-like 2 (LOXL2) protein.

16. The method of claim 10, wherein the anti-LOXL2 antibody is administered intravenously or subcutaneously.

17. The method of claim 10, wherein the anti-LOXL2 antibody is administered at 75 or 125 mg.

18. The method of claim 10, wherein the anti-LOXL2 antibody is administered once a week.

19. The method of claim 9, wherein the ASK1 inhibitor and the LOXL2 inhibitor are administered together.

20. The method of claim 9, wherein the ASK1 inhibitor and the LOXL2 inhibitor are administered separately.

21. A pharmaceutical composition comprising a therapeutically effective amount of an ASK1 inhibitor and a therapeutically effective amount of a LOXL2 inhibitor.

22. A kit comprising a therapeutically effective amount of an ASK1 inhibitor and a therapeutically effective amount of a LOXL2 inhibitor.


SEQUENCE LISTING (16 sequences)

SEQ ID NO: 1 - protein, 219 aa, Unknown
  anti-LOXL2 binding epitope
VRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACS

SEQ ID NO: 2 - protein, 116 aa, Unknown
  anti-LOXL2 antibody heavy chain variable region complementarity determining region
QVQLVQSGAELKKPGASVKVSCKASGYAFTYYLIEWVKQAPGQGLEWIGVINPGSGGTNYNEKFKGRATLTADKSTSTAYMELSSLRSEDSAVYFCARNWMNFDYWGQGTTVTVSS

SEQ ID NO: 3 - protein, 116 aa, Unknown
  anti-LOXL2 antibody heavy chain variable region complementarity determining region
QVQLVQSGAEVKKPGASVKVSCKASGYAFTYYLIEWVRQAPGQGLEWIGVINPGSGGTNYNEKFKGRATLTADKSTSTAYMELSSLRSEDTAVYFCARNWMNFDYWGQGTTVTVSS

SEQ ID NO: 4 - protein, 116 aa, Unknown
  anti-LOXL2 antibody heavy chain variable region complementarity determining region
QVQLVQSGAEVKKPGASVKVSCKASGYAFTYYLIEWVRQAPGQGLEWIGVINPGSGGTNYNEKFKGRATITADKSTSTAYMELSSLRSEDTAVYFCARNWMNFDYWGQGTTVTVSS

SEQ ID NO: 5 - protein, 116 aa, Unknown
  anti-LOXL2 antibody heavy chain variable region complementarity determining region
QVQLVQSGAEVKKPGASVKVSCKASGYAFTYYLIEWVRQAPGQGLEWIGVINPGSGGTNYNEKFKGRVTITADKSTSTAYMELSSLRSEDTAVYYCARNWMNFDYWGQGTTVTVSS

SEQ ID NO: 6 - protein, 112 aa, Unknown
  anti-LOXL2 antibody light chain variable region complementarity determining region
DIVMTQTPLSLSVTPGQPASISCRSSKSLLHSNGNTYLYWFLQKPGQSPQFLIYRMSNLASGVPDRFSGSGSGTAFTLKISRVEAEDVGVYYCMQHLEYPYTFGGGTKVEIK

SEQ ID NO: 7 - protein, 112 aa, Unknown
  anti-LOXL2 antibody light chain variable region complementarity determining region
DIVMTQTPLSLSVTPGQPASISCRSSKSLLHSNGNTYLYWFLQKPGQSPQFLIYRMSNLASGVPDRFSGSGSGTDFTLKISRVEAEDVGVYYCMQHLEYPYTFGGGTKVEIK

SEQ ID NO: 8 - protein, 112 aa, Unknown
  anti-LOXL2 antibody light chain variable region complementarity determining region
DIVMTQTPLSLSVTPGQPASISCRSSKSLLHSNGNTYLYWYLQKPGQSPQFLIYRMSNLASGVPDRFSGSGSGTDFTLKISRVEAEDVGVYYCMQHLEYPYTFGGGTKVEIK

SEQ ID NO: 9 - protein, 10 aa, Unknown
  anti-LOXL2 antibody heavy chain variable region complementarity determining region 1
GYAFTYYLIE

SEQ ID NO: 10 - protein, 17 aa, Unknown
  anti-LOXL2 antibody heavy chain variable region complementarity determining region 2
VINPGSGGTNYNEKFKG

SEQ ID NO: 11 - protein, 7 aa, Unknown
  anti-LOXL2 antibody heavy chain variable region complementarity determining region 3
NWMNFDY

SEQ ID NO: 12 - protein, 16 aa, Unknown
  anti-LOXL2 antibody light chain variable region complementarity determining region 1
RSSKSLLHSNGNTYLY

SEQ ID NO: 13 - protein, 7 aa, Unknown
  anti-LOXL2 antibody light chain variable region complementarity determining region 2
RMSNLAS

SEQ ID NO: 14 - protein, 9 aa, Unknown
  anti-LOXL2 antibody light chain variable region complementarity determining region 3
MQHLEYPYT

SEQ ID NO: 15 - protein, 135 aa, Unknown
  anti-LOXL2 antibody AB0023 heavy chain variable region
MEWSRVFIFLLSVTAGVHSQVQLQQSGAELVRPGTSVKVSCKASGYAFTYYLIEWVKQRPGQGLEWIGVINPGSGGTNYNEKFKGKATLTADKSSSTAYMQLSSLTSDDSAVYFCARNWMNFDYWGQGTTLTVSS

SEQ ID NO: 16 - protein, 132 aa, Unknown
  anti-LOXL2 antibody AB0023 light chain variable region
MRCLAEFLGLLVLWIPGAIGDIVMTQAAPSVSVTPGESVSISCRSSKSLLHSNGNTYLYWFLQRPGQSPQFLIYRMSNLASGVPDRFSGSGSGTAFTLRISRVEAEDVGVYYCMQHLEYPYTFGGGTKLEIK